(19) United States
(12) Patent Application Publication
(10) Pub. No.: US 2010/0196451 A1
(43) Pub. Date: Aug. 5, 2010

(54) Vaccines Containing Non-Live Antigenic Vectors

(75) Inventors: Nathalie Marie-Josephe Garcon, Rixensart (BE); Emmanuel Jules Hanon, Rixensart (BE)

Correspondence Address: GLAXOSMITHKLINE;CORPORATE INTELLECTUAL PROPERTY, MAI B482, FIVE MOORE DR., PO BOX 13398, RESEARCH TRIANGLE PARK, NC, 27709-3398, US

(73) Assignee: GlaxoSmithKline Biologicals

(21) Appl. No.: 12/095,539

(22) PCT Filed: Nov. 28, 2006
(86) PCT No.: PCT/EP06/11470

§ 371 Date: May 30, 2008

(30) Foreign Application Priority Data
Nov. 30, 2005 (GB) 0524408.2

(51) Int. Cl. A61K 9/127; A61K 39/108; A61K 39/10; A61K 39/02; A61K 39/29; A61P 37/04

(52) U.S. Cl. 424/450; 424/236.1; 424/241.1; 424/240.1; 424/234.1; 424/228.1

(57) ABSTRACT

The present invention provides a vaccine composition comprising a non-live vector which targets the MHC class I pathway derived from a bacterial toxin or an immunologically functional derivative thereof but excluding those which bind the Gb3 receptor complexed with at least one first antigen and further comprising at least one second antigen (which may be the same or different as the first antigen) and an adjuvant.

The present invention provides improved vaccine compositions, methods for making them and their use in medicine. In particular the present invention provides adjuvanted vaccine compositions which comprise an agent which can improve MHC class I presentation of an antigen complexed to a first antigen, and a second antigen which may be the same or different from the first antigen, said composition being formulated with an adjuvant

The development of vaccines which require a predominant induction of a cellular response remains a challenge. Because CD8+ T cells, the main effector cells of the cellular immune response, recognise antigens that are synthesized in pathogen-infected cells, successful vaccination requires the synthesis of immunogenic antigens in cells of the vaccine. This can be achieved with live-attenuated vaccines, however they also present significant limitations. First, there is a risk of infection, either when vaccines are immunosuppressed, or when the pathogen itself can induce immunosuppression (e.g. Human Immunodeficiency Virus). Second, some pathogens are difficult or impossible to grow in cell culture (e.g. Hepatitis C Virus). Other existing vaccines such as inactivated whole-cell vaccines or alum adjuvanted, recombinant protein subunit vaccines are notably poor inducers of CD8 responses.

For these reasons, alternative approaches are being developed: live vectored vaccines, plasmid DNA vaccines, synthetic peptides or specific adjuvants. Live vectored vaccines are good at inducing a strong cellular response but pre-existing (e.g. adenovirus) or vaccine-induced immunity against the vector may jeopardize the efficiency of additional vaccine dose (Casimiro et al, JOURNAL OF VIROLOGY, June 2003, p. 6305-6313). Plasmid DNA vaccines also can induce a cellular response (Casimiro et al, JOURNAL OF VIROLOGY, June 2003, p. 6305-6313) but it remains weak in humans (Mc Conkey et al, Nature Medicine 9, 729-735, 2003) and the antibody response is very poor. In addition, synthetic peptides are currently being evaluated in clinical trials (Khong et al, J Immunother 2004; 27:472-477), but the efficacy of such vaccines encoding a limited number of T cell epitopes may be hampered by the appearance of vaccine escape mutants or by the necessity of first selecting for HLA-matched patients.

Alternative approaches aimed at improving MHC class I presentation have also been described, based on antigen delivery using non-live vectors. Some non-live vectors are derived from bacterial toxins, for example Anthrax LFn toxin (Ballard et al (1996) PNAS USA 93 pp 12531-12534), B. pertussis adenylate cyclase toxin (Fayolle et al (1996) J. Immunology 156 p 4697-4706) or Pseudomonas Exotoxin A (Donnelly et al PNAS USA (1993) 90 pp 3530-3534).

The limitations of vaccine antigens and delivery systems justify the search for new vaccine compositions. The present inventors have found that the inclusion of adjuvants in compositions comprising non-live vectors aimed at improving MHC class I presentation can have a beneficial effect on the resulting immune response, in particular CD8 specific responses. It is thought that this beneficial effect occurs because of the combination of the activation of the immune response given by an adjuvant with the correct delivery of an antigen provided by an agent which targets the MHC1 pathway.

In addition, there would be advantages to a vaccine composition that could activate, as discussed above, CD8 responses whilst at the same time activating CD4 responses or generating a specific antibody response.

Therefore the present invention provides a vaccine composition comprising a non-live vector aimed at improving MHC class I presentation or an immunologically functional derivative thereof but excluding those which bind the Gb3 receptor, complexed with a first antigen and further comprising one or more second antigens which may be the same or different to the first antigen and an adjuvant.

The present inventors have also found that the inclusion of the same antigen in both free and complexed form enables the activation of both cellular and humoral immunity to the antigen. The present inventors have further found that the inclusion of one antigen in complexed form and one antigen in free form enables the activation of cellular and humoral immunity to both antigens thereby providing a complete immune response. The present inventors have also found that the inclusion of adjuvant in compositions comprising antigen complexed to a non-live vector which targets MHC class I presentation and further comprising free antigen has a beneficial effect on the immune response to, in particular, the complexed antigen.

The term “non-live vector” as used herein is defined as an antigen delivery agent which targets MHC class I presentation. This term is not intended to encompass replicating vectors, such as attenuated viruses, bacteria, or plasmid DNA. The non-live vector is derived from a bacterial toxin, that is the non-live vector is a detoxified bacterial toxin, subunit or immunologically functional equivalent.

In the context of the invention, the word toxin is intended to mean toxins that have been detoxified such that they are no longer toxic to humans, or a toxin subunit or fragment thereof that are substantially devoid of toxic activity in humans.

Preferred non-live vectors based on detoxified toxins are the amino terminal domain of the anthrax lethal factor (LF), P. aeruginosa exotoxin A, the B subunit from E. coli labile toxin (LT), and the adenylate cyclase A from B. pertussis. In one embodiment, the non-live vector is the B subunit from E. coli labile toxin type I (LTI) In one embodiment, the non-live vector is derived from a toxin which is a family of the AB5 family, for example LT2, the cholera toxin (CT), the Bordatella Pertussis toxin (PT) as well as the recently identified subtilase cytotoxins. (Paton et al, J Exp Med 2004, Vol 200 pp 35-46).

The amino-terminal domain from B. Anthracis (anthrax) LF is known as LFn. It is the N-terminal 255 amino acids of LF. LF has been found to contain the information necessary for binding to protective antigen (PA) and mediating translocation. The domain alone lacks lethal potential, that depends on the putatively enzymatic carboxyl-terminal moiety (Arora and Leppla (1993) J. Biol Chem 268 pp 3334-3341). In addition, it was recently found that a fusion protein of the LFn domain with a foreign antigen can induce CD8 T cell immune responses even in the absence of PA (Kushner et al (2003), PNAS 100 pp 6652-6657) suggesting that LFn may be used without PA as a carrier to deliver antigens into the cytosol.

The labile toxin (LT1) of E. coli consists of two subunits, a pentameric B subunit and a monomeric A subunit. The A subunit is responsible for toxicity, whilst the B subunit is responsible for transport into the cell. LT binds the GM1 ganglioside receptor.

Donnelly et al (Supra) demonstrate that the toxic domain may be removed from P. aeroginosa and the remainder of the toxin may still mediate transport of an antigen into the cell. In addition, deletion of amino acids from the full-length toxin does not impair its ability to access to the cytosol but renders it nontoxic since this mutation eliminates the ADP-ribosylating activity. Based on this mutant, chimeras can be constructed that encode antigenic sequences of various sizes (Fitzgerald, J Biol Chem, Vol. 273, Issue 16, 9951-9958, Apr. 17, 1998.

The adenylate cyclase toxin binds the CD11b receptor at the surface of dendritic cells. Recombinant toxoids bearing CD8⁺ T-cell epitopes are able to induce specific CTL responses in mice and protection against experimental tumours has been demonstrated (Fayolle et al, J Immunol 1999, 162 pp 4157-4162). Surface presentation of the delivered epitopes occurs via the classical MHC class I pathway

An immunologically functional equivalent thereof of a particular non-live vector derived from a bacterial toxin is herein defined as proteins that retain at least 50% amino acid sequence identity to that non-live protein vector, for example 60, 70, 80, 90, 95 or 96, 97, 98 or 99% identity, and are still able to target an antigen to the MHC class I pathway. For example, amino acid deletions, insertions, or substitutions may be made that do not affect the function of the non-live vector. Where the receptor for a particular toxin is known, immunologically functional equivalents are defined as proteins that retain at least 50% amino acid sequence identity to the bacterial toxin, and are still able to bind that receptor. For example, in the case of the B subunit of Lt, an immunologically functional equivalent is defined as a protein which retains at least 50% sequence identity to the B subunit of Lt, and is still able to bind the GM1 ganglioside receptor. Such immunologically functional equivalents may themselves be bacterial toxins, for example the B subunit of Cholera toxin (CT) is an immunologically functional equivalent of LT. Whether a vector or equivalent binds the GM1 receptor may be determined, for example, by following the protocol set out in example 1.1 below.

Other vectors that may be used in the present invention may be derived by using a receptor or receptor mimic that a bacterial toxin is known to bind to for screening a phage-display library (a technique sometimes known as biopanning). Such a technique would provide peptides (for example up to 20 amino acids or so in length) that could bind the same receptor as the bacterial toxin, but would have little or no sequence similarity to the toxin. This technique has been shown to be an effective way of generating peptides that bind to the GB3 receptor (Miura et al Biochimica et Biphysica Acta 1673 (2004) pp 131-138) and the GM1 receptor (Matsubara et al FEBS letters 456 (1999) 253-256. It is likely that such peptides could act as vectors in the same way as the bacterial toxins which bind to the same receptors. Such peptides are considered to fall within the definition “vector derived from a bacterial toxin” as they are derived by screening at the same receptor as that that the bacterial toxin binds to. In one embodiment, however, the vector of the invention that is “derived from a bacterial toxin” is actually a bacterial toxin or an immunologically functional equivalent thereof.

Not included within the scope of the present invention are those non-live vectors or immunologically functional equivalents thereof which are able to bind the Gb3 receptor. Whether a vector or equivalent binds the Gb3 receptor may be determined, for example, by following the protocol set out in example 1.4 below.

The compositions of the invention are capable of improving a CD8 specific immune response to the antigen complexed to a protein of the invention. Improvement is measured by looking at the response to a composition of the invention comprising a first antigen complexed to a protein of the invention and a second antigen and further comprising an adjuvant when compared to the response to a composition comprising a first antigen complexed to a protein of the invention and a second antigen with no adjuvant, or the response to a formulation comprising a first and second antigen with adjuvant. Improvement may be defined as an increase in the level of the immune response, the generation of an equivalent immune response with a lower dose of antigen, an increase in the quality of the immune response, an increase in the persistency of the immune response, or any combination of the above. Such an improvement may be seen following a first immunization, and/or may be seen following subsequent immunizations.

Particular adjuvants are those selected from the group of metal Salts, oil in water emulsions, Toll like receptors ligand, (in particular Toll like receptor 2 ligand, Toll like receptor 3 ligand, Toll like receptor 4 ligand, Toll like receptor 7 ligand, Toll like receptor 8 ligand and Toll like receptor 9 ligand), saponins or combinations thereof. In one embodiment, the adjuvant does not comprise a metal salt as sole adjuvant. In another embodiment, the adjuvant does not comprise a metal salt. In one embodiment, the toll like receptor ligand is a receptor agonist. In another embodiment, the toll like receptor ligand is a receptor antagonist. The term “ligand” as used throughout the specification and the claims is intended to mean an entity that can bind to the receptor and have an effect, either to upregulate or downregulate the activity of the receptor.

The adjuvant is preferably selected from the group: a saponin, lipid A or a derivative thereof, an immunostimulatory oligonucleotide, an alkyl glucosaminide phosphate, or combinations thereof. A further preferred adjuvant is a metal salt in combination with another adjuvant. It is preferred that the adjuvant is a Toll like receptor ligand in particular an ligand of a Toll like receptor 2, 3, 4, 7, 8 or 9, or a saponin, in particular Qs21. It is further preferred that the adjuvant system comprises two or more adjuvants from the above list. In particular the combinations preferably contain a saponin (in particular Qs21) adjuvant and/or a Toll like receptor 9 ligand such as a immunostimulatory oligonucleotide containing CpG or other immunostimulatory motifs such as CpR where R is a non-natural guanosine nucleotide. Other preferred combinations comprise a saponin (in particular QS21) and a Toll like receptor 4 ligand such as monophosphoryl lipid A or its 3 deacylated derivative, 3 D-MPL, or a saponin (in particular QS21) and a Toll like receptor 4 ligand such as an alkyl glucosaminide phosphate. Other preferred combinations comprise a TLR 3 or 4 ligand in combination with a TLR 8 or 9 ligand.

Particularly preferred adjuvants are combinations of 3D-MPL and QS21 (EP 0 671 948 B1), oil in water emulsions comprising 3D-MPL and QS21 (WO 95/17210, WO 98/56414), or 3D-MPL formulated with other carriers (EP 0 689 454 B1). Other preferred adjuvant systems comprise a combination of 3 D MPL, QS21 and a CpG oligonucleotide as described in U.S. Pat. No. 6,558,670, U.S. Pat. No. 6,544,518.

In an embodiment the adjuvant is a Toll like receptor (TLR) 4 ligand, preferably an ligand such as a lipid A derivative particularly monophosphoryl lipid A or more particularly 3 Deacylated monophoshoryl lipid A (3 D-MPL).

3 D-MPL is sold under the trademark MPL® by GSK biologicals and primarily promotes CD4+ T cell responses with an IFN-g (Th1) phenotype. It can be produced according to the methods disclosed in GB 2 220 211 A. Chemically it is a mixture of 3-deacylated monophosphoryl lipid A with 3, 4, 5 or 6 acylated chains. Preferably in the compositions of the present invention small particle 3 D-MPL is used. Small particle 3 D-MPL has a particle size such that it may be sterile-filtered through a 0.22 μm filter. Such preparations are described in International Patent Application No. WO 94/21292. Synthetic derivatives of lipid A are known and thought to be TLR 4 ligands including, but not limited to:
OM174 (2-deoxy-6-o-[2-deoxy-2-[(R)-3-dodecanoyloxytetra-decanoylamino]-4-O-phosphono-β-D-glucopyranosyl]-2-[(R)-3-hydroxytetradecanoylamino]-α-D-glucopyranosyldihydrogenphosphate), (WO 95/14026)
OM 294 DP (3S,9R)-3-[(R)-dodecanoyloxytetradecanoylamino]-4-oxo-5-aza-9(R)-[(R)-3-hydroxytetradecanoylamino]decan-1,10-diol,1,10-bis(dihydrogenophosphate) (WO99/64301 and WO 00/0462)
OM 197 MP-Ac DP (3S-,9R)-3-[(R)-dodecanoyloxytetradecanoylamino]-4-oxo-5-aza-9-[(R)-3-hydroxytetradecanoylamino]decan-1,10-diol,1-dihydrogenophosphate 10-(6-aminohexanoate) (WO 01/46127)

Other TLR4 ligands which may be used are alkyl Glucosaminide phosphates (AGPs) such as those disclosed in WO9850399 or U.S. Pat. No. 6,303,347 (processes for preparation of AGPs are also disclosed), or pharmaceutically acceptable salts of AGPs as disclosed in U.S. Pat. No. 6,764,840. Some AGPs are TLR4 agonists, and some are TLR4 antagonists. Both are thought to be useful as adjuvants.

Another preferred immunostimulant for use in the present invention is Quil A and its derivatives. Quil A is a saponin preparation isolated from the South American tree Quilaja Saponaria Molina and was first described as having adjuvant activity by Dalsgaard et al. in 1974 (“Saponin adjuvants”, Archiv. für die gesamte Virusforschung, Vol. 44, Springer Verlag, Berlin, p 243-254). Purified fragments of Quil A have been isolated by HPLC which retain adjuvant activity without the toxicity associated with Quil A (EP 0 362 278), for example QS7 and QS21 (also known as QA7 and QA21). QS-21 is a natural saponin derived from the bark of Quillaja saponaria Molina which induces CD8+ cytotoxic T cells (CTLs), Th1 cells and a predominant IgG2a antibody response and is a preferred saponin in the context of the present invention.

Particular formulations of QS21 have been described which are particularly preferred, these formulations further comprise a sterol (WO96/33739). The saponins forming part of the present invention may be separate in the form of micelles, mixed micelles (preferentially, but not exclusively with bile salts) or may be in the form of ISCOM matrices (EP 0 109 942 B1), liposomes or related colloidal structures such as worm-like or ring-like multimeric complexes or lipidic/layered structures and lamellae when formulated with cholesterol and lipid, or in the form of an oil in water emulsion (for example as in WO 95/17210). The saponins may preferably be associated with a metallic salt, such as aluminium hydroxide or aluminium phosphate (WO 98/15287).

Preferably, the saponin is presented in the form of a liposome, ISCOM or an oil in water emulsion.

Immunostimulatory oligonucleotides or any other Toll-like receptor (TLR) 9 ligand may also be used. The preferred oligonucleotides for use in adjuvants or vaccines of the present invention are CpG containing oligonucleotides, preferably containing two or more dinucleotide CpG motifs separated by at least three, more preferably at least six or more nucleotides. A CpG motif is a Cytosine nucleotide followed by a Guanine nucleotide. The CpG oligonucleotides of the present invention are typically deoxynucleotides. In a preferred embodiment the internucleotide in the oligonucleotide is phosphorodithioate, or more preferably a phosphorothioate bond, although phosphodiester and other internucleotide bonds are within the scope of the invention. Also included within the scope of the invention are oligonucleotides with mixed internucleotide linkages. Methods for producing phosphorothioate oligonucleotides or phosphorodithioate are described in U.S. Pat. No. 5,666,153, U.S. Pat. No. 5,278,302 and WO95/26204.

Examples of preferred oligonucleotides have the following sequences. The sequences preferably contain phosphorothioate modified internucleotide linkages.

| | |
|---|---|
| OLIGO 1 (SEQ ID NO: 1): | |
| TCC ATG ACG TTC CTG ACG TT | (CpG 1826) |
| OLIGO 2 (SEQ ID NO: 2): | |
| TCT CCC AGC GTG CGC CAT | (CpG 1758) |
| OLIGO 3 (SEQ ID NO: 3): | |
| AGC GAT GAC GTC GCC GGT GAC GGC ACC ACG | |
| OLIGO 4 (SEQ ID NO: 4): | |
| TCG TCG TTT TGT CGT TTT GTC GTT | (CpG 2006) |
| OLIGO 5 (SEQ ID NO: 5): | |
| TCC ATG ACG TTC CTG ATG CT | (CpG 1668) |
| OLIGO 6 (SEQ ID NO: 6): | |
| TCG ACG TTT TCG GCG CGC GCC G | (CpG 5456) |

Alternative CpG oligonucleotides may comprise the preferred sequences above in that they have inconsequential deletions or additions thereto.

Alternative immunostimulatory oligonucleotides may comprise modifications to the nucleotides. For example, WO0226757 and WO03507822 disclose modifications to the C and G portion of a CpG containing immunostimulatory oligonucleotides.

The immunostimulatory oligonucleotides utilised in the present invention may be synthesized by any method known in the art (for example see EP 468520).

Conveniently, such oligonucleotides may be synthesized utilising an automated synthesizer.

Examples of a TLR 2 ligand include peptidoglycan or lipoprotein. Imidazoquinolines, such as Imiquimod and Resiquimod are known TLR7 ligands. Single stranded RNA is also a known TLR ligand (TLR8 in humans and TLR7 in mice), whereas double stranded RNA and poly IC (polyinosinic-polycytidylic acid—a commercial synthetic mimetic of viral RNA). are exemplary of TLR 3 ligands. 3D-MPL is an example of a TLR4 ligand whilst CPG is an example of a TLR9 ligand

The non-live vector derived from a bacterial toxin or immunologically functional equivalent thereof and the antigen are complexed together. By complexed is meant that the non-live vector derived from a bacterial toxin or immunologically functional equivalent thereof and the first antigen are physically associated, for example via an electrostatic or hydrophobic interaction or a covalent linkage. In a preferred embodiment the non-live vector derived from a bacterial toxin or immunologically functional equivalent thereof are covalently linked either as a fusion protein or chemically coupled, for example via a cysteine residue. In embodiments of the invention more than one antigen is linked to each non-live vector or immunologically functional equivalent thereof such as 2, 3, 4, 5 6 antigen molecules per vector. When more than one antigen is present, these antigens may all be the same, one or more may be different to the others, or all the antigens may be different to each other.

The antigens themselves may be a peptide, or a protein encompassing one or more epitopes of interest. It is a preferred embodiment that the first antigen is selected such that when formulated in the manner contemplated by the invention it provides immunity against intracellular pathogens such as HIV, tuberculosis, Chlamydia, HBV, HCV, and Influenza The present invention also finds utility with antigens which can raise relevant immune responses against benign and proliferative disorders such as Cancers.

Preferably the vaccine formulations of the present invention contain an antigen or antigenic composition capable of eliciting an immune response against a human pathogen, which antigen or antigenic composition is derived from HIV-1, (such as gag or fragments thereof, such as p24, tat, nef, envelope such as gp120 or gp160, or fragments of any of these), human herpes viruses, such as gD or derivatives thereof or Immediate Early protein such as ICP27 from HSV1 or HSV2, cytomegalovirus ((esp Human) (such as gB or derivatives thereof), Rotaviral antigen, Epstein Barr virus (such as gp350 or derivatives thereof), Varicella Zoster Virus (such as gpI, II and IE63), or from a hepatitis virus such as hepatitis B virus (for example Hepatitis B Surface antigen or a derivative thereof), or antigens from hepatitis A virus, hepatitis C virus and hepatitis E virus, or from other viral pathogens, such as paramyxoviruses: Respiratory Syncytial virus (such as F G and N proteins or derivatives thereof), parainfluenza virus, measles virus, mumps virus, human papilloma viruses (for example HPV 6, 11, 16, 18,) flaviviruses (e.g. Yellow Fever Virus, Dengue Virus, Tick-borne encephalitis virus, Japanese Encephalitis Virus) or Influenza virus purified or recombinant proteins thereof, such as HA, NP, NA, or M proteins, or combinations thereof), or derived from bacterial pathogens such as Neisseria spp, including N. gonorrhea and N. meningitidis (for example, transferrin-binding proteins, lactoferrin binding proteins, PilC, adhesins); S. pyogenes (for example M proteins or fragments thereof, C5A protease,), S. agalactiae, S. mutans; H. ducreyi; Moraxella spp, including M. catarrhalis, also known as Branhamella catarrhalis (for example high and low molecular weight adhesins and invasins); Bordetella spp, including B. pertussis (for example pertactin, pertussis toxin or derivatives thereof, filamenteous hemagglutinin, adenylate cyclase, fimbriae), B. parapertussis and B. bronchiseptica; Mycobacterium spp., including M. tuberculosis (for example ESAT6, Antigen 85A, -B or -C), M. bovis, M. leprae, M. avium, M. paratuberculosis, M. smegmatis; Legionella spp, including L. pneumophila; Escherichia spp, including enterotoxic E. coli (for example colonization factors, heat-labile toxin or derivatives thereof, heat-stable toxin or derivatives thereof), enterohemorragic E. coli, enteropathogenic E. coli Vibrio spp, including V. cholera (for example cholera toxin or derivatives thereof); Shigella spp, including S. sonnei, S. dysenteriae, S. flexnerii; Yersinia spp, including Y. enterocolitica (for example a Yop protein), Y. pestis, Y. pseudotuberculosis; Campylobacter spp, including C. jejuni (for example toxins, adhesins and invasins) and C. coli; Salmonella spp, including S. typhi, S. paratyphi, S. choleraesuis, S. enteritidis; Listeria spp., including L. monocytogenes; Helicobacter spp, including H. pylori (for example urease, catalase, vacuolating toxin); Pseudomonas spp, including P. aeruginosa; Staphylococcus spp., including S. aureus, S. epidermidis; Enterococcus spp., including E. faecalis, E. faecium; Clostridium spp., including C. tetani (for example tetanus toxin and derivative thereof), C. botulinum (for example botulinum toxin and derivative thereof), C. difficile (for example clostridium toxins A or B and derivatives thereof); Bacillus spp., including B. anthracis (for example botulinum toxin and derivatives thereof); Corynebacterium spp., including C. diphtheriae (for example diphtheria toxin and derivatives thereof); Borrelia spp., including B. burgdorferi (for example OspA, OspC, DbpA, DbpB), B. garinii (for example OspA, OspC, DbpA, DbpB), B. afzelii (for example OspA, OspC, DbpA, DbpB), B. andersonii (for example OspA, OspC, DbpA, DbpB), B. hermsii; Ehrlichia spp., including E. equi and the agent of the Human Granulocytic Ehrlichiosis; Rickettsia spp, including R. rickettsii; Chlamydia spp., including C. trachomatis (for example MOMP, heparin-binding proteins), C. pneumoniae (for example MOMP, heparin-binding proteins), C. psittaci; Leptospira spp., including L. interrogans; Treponema spp., including T. pallidum (for example the rare outer membrane proteins), T. denticola, T. hyodysenteriae; or derived from parasites such as Plasmodium spp., including P. falciparum; Toxoplasma spp., including T. gondii (for example SAG2, SAG3, Tg34); Entamoeba spp., including E. histolytica; Babesia spp., including B. microti; Trypanosoma spp., including T. cruzi; Giardia spp., including G. lamblia; Leshmania spp., including L. major; Pneumocystis spp., including P. carinii; Trichomonas spp., including T. vaginalis; Schisostoma spp., including S. mansoni, or derived from yeast such as Candida spp., including C. albicans; Cryptococcus spp., including C. neoformans.

Other preferred specific antigens for M. tuberculosis are for example Tb Ra12, Tb H9, Tb Ra35, Tb38-1, Erd 14, DPV, MTI, MSL, mTTC2 and hTCC1 (WO 99/51748). Proteins for M. tuberculosis also include fusion proteins and variants thereof where at least two, preferably three polypeptides of M. tuberculosis are fused into a larger protein. Preferred fusions include Ra12-TbH9-Ra35, Erd14-DPV-MTI, DPV-MTI-MSL, Erd14-DPV-MTI-MSL-mTCC2, Erd14-DPV-MTI-MSL, DPV-MTI-MSL-mTCC2, TbH9-DPV-MTI (WO 99/51748).

Most preferred antigens for Chlamydia include for example the High Molecular Weight Protein (HMW) (WO 99/17741), ORF3 (EP 366 412), and putative membrane proteins (Pmps). Other Chlamydia antigens of the vaccine formulation can be selected from the group described in WO 99/28475.

Preferred bacterial vaccines comprise antigens derived from Streptococcus spp, including S. pneumoniae (for example, PsaA, PspA, streptolysin, choline-binding proteins) and the protein antigen Pneumolysin (Biochem Biophys Acta, 1989, 67, 1007; Rubins et al., Microbial Pathogenesis, 25, 337-342), and mutant detoxified derivatives thereof (WO 90/06951; WO 99/03884). Other preferred bacterial vaccines comprise antigens derived from Haemophilus spp., including H. influenzae type B, non typeable H. influenzae, for example OMP26, high molecular weight adhesins, P5, P6, protein D and lipoprotein D, and fimbrin and fimbrin derived peptides (U.S. Pat. No. 5,843,464) or multiple copy variants or fusion proteins thereof.

Derivatives of Hepatitis B Surface antigen are well known in the art and include, inter alia, those PreS1, PreS2 S antigens set forth described in European Patent applications EP-A-414 374; EP-A-0304 578, and EP 198-474. In one preferred aspect the vaccine formulation of the invention comprises the HIV-1 antigen, gp120, especially when expressed in CHO cells. In a further embodiment, the vaccine formulation of the invention comprises gD2t as hereinabove defined.

In a preferred embodiment of the present invention the vaccine compositions comprise antigen derived from the Human Papilloma Virus (HPV) considered to be responsible for genital warts (HPV 6 or HPV 11 and others), and the HPV viruses responsible for cervical cancer (HPV16, HPV18 and others).

Particularly preferred forms of genital wart prophylactic, or therapeutic, vaccine comprise L1 protein, and fusion proteins comprising one or more antigens selected from the HPV proteins E1, E2, E5, E6, E7, L1, and L2.

The most preferred forms of fusion protein are: L2E7 as disclosed in WO 96/26277, and proteinD(1/3)-E7 disclosed in WO99/10375.

A preferred HPV cervical infection or cancer, prophylaxis or therapeutic vaccine composition may comprise HPV 16 or 18 antigens.

Particularly preferred HPV 16 antigens comprise the early proteins E6 or E7 in fusion with a protein D carrier to form Protein D-E6 or E7 fusions from HPV 16, or combinations thereof; or combinations of E6 or E7 with L2 (WO 96/26277).

Alternatively the HPV 16 or 18 early proteins E6 and E7, may be presented in a single molecule, preferably a Protein D-E6/E7 fusion. Such vaccine may optionally contain either or both E6 and E7 proteins from HPV 18, preferably in the form of a Protein D-E6 or Protein D-E7 fusion protein or Protein D E6/E7 fusion protein.

The vaccine of the present invention may additionally comprise antigens from other HPV strains, preferably from strains HPV 31 or 33.

Vaccine compositions of the present invention further comprise antigens derived from parasites that cause Malaria, for example, antigens from Plasmodia falciparum including circumsporozoite protein (CS protein), RTS,S, MSP1, MSP3, LSA1, LSA3, AMA1 and TRAP. RTS is a hybrid protein comprising substantially all the C-terminal portion of the circumsporozoite (CS) protein of P. falciparum linked via four amino acids of the preS2 portion of Hepatitis B surface antigen to the surface (S) antigen of hepatitis B virus. Its full structure is disclosed in International Patent Application No. PCT/EP92/02591, published under Number WO 93/10152 claiming priority from UK patent application No. 9124390.7. When expressed in yeast RTS is produced as a lipoprotein particle, and when it is co-expressed with the S antigen from HBV it produces a mixed particle known as RTS,S. TRAP antigens are described in International Patent Application No. PCT/GB89/00895, published under WO 90/01496. Plasmodia antigens that are likely candidates to be components of a multistage Malaria vaccine are P. falciparum MSP1, AMA1, MSP3, EBA, GLURP, RAP1, RAP2, Sequestrin, PfEMP1, Pf332, LSA1, LSA3, STARP, SALSA, PfEXP1, Pfs25, Pfs28, PFS27/25, Pfs16, Pfs48/45, Pfs230 and their analogues in Plasmodium spp. One embodiment of the present invention is a malaria vaccine wherein the antigen preparation comprises RTS,S or CS protein or a fragment thereof such as the CS portion of RTS,S, in combination with one or more further malarial antigens, either or both of which may be attached to the Shiga toxin B subunit in accordance with the invention. The one or more further malarial antigens may be selected for example from the group consisting of MPS1, MSP3, AMA1, LSA1 or LSA3.

The formulations may also contain an anti-tumour antigen and be useful for the immunotherapeutic treatment of cancers. For example, the adjuvant formulation finds utility with tumour rejection antigens such as those for prostrate, breast, colorectal, lung, pancreatic, renal or melanoma cancers. Exemplary antigens include MAGE 1 and MAGE 3 or other MAGE antigens (for the treatment of melanoma), PRAME, BAGE, or GAGE (Robbins and Kawakami, 1996, Current Opinions in Immunology 8, pps 628-636; Van den Eynde et al., International Journal of Clinical & Laboratory Research (submitted 1997); Correale et al. (1997), Journal of the National Cancer Institute 89, p293. Indeed these antigens are expressed in a wide range of tumour types such as melanoma, lung carcinoma, sarcoma and bladder carcinoma. Other tumour-specific antigens are suitable for use with the adjuvants of the present invention and include, but are not restricted to tumour-specific gangliosides, Prostate specific antigen (PSA) or Her-2/neu, KSA (GA733), PAP, mammaglobin, MUC-1, carcinoembryonic antigen (CEA) or p501 S (prostein). Accordingly in one aspect of the present invention there is provided a vaccine comprising an adjuvant composition according to the invention and a tumour rejection antigen.

It is a particularly preferred aspect of the present invention that the vaccines comprise a tumour antigen such as prostrate, breast, colorectal, lung, pancreatic, renal, ovarian or melanoma cancers. Accordingly, the formulations may contain tumour-associated antigen, as well as antigens associated with tumour-support mechanisms (e.g. angiogenesis, tumour invasion). Additionally, antigens particularly relevant for vaccines in the therapy of cancer also comprise Prostate-specific membrane antigen (PSMA), p501S (prostein), Prostate Stem Cell Antigen (PSCA), tyrosinase, survivin, NY-ESO1, prostase, PS108 (WO 98/50567), RAGE, LAGE, HAGE. Additionally said antigen may be a self peptide hormone such as whole length Gonadotrophin hormone releasing hormone (GnRH, WO 95/20600), a short 10 amino acid long peptide, useful in the treatment of many cancers, or in immunocastration.

Vaccines of the present invention may be used for the prophylaxis or therapy of allergy. Such vaccines would comprise allergen specific antigens, for example Der p1

In one aspect of the invention, the vaccine compositions of the invention comprise more than one different antigen, wherein at least one antigen is complexed to a protein of the invention. Such compositions would be useful to raise immune responses wherein the antigen that is complexed to the protein of the invention is an internal antigen from a pathogen and as such would not generally generate an antibody response and therefore needs to be directed into the MHC class I presenting pathway. In addition, the composition further comprises at least one second antigen that is not complexed to a protein of the invention. In a preferred aspect, this second non complexed antigen can raise an antibody response or can be directed through the MHC class II presenting pathway. This dual approach ensures that as many different arms of the immune system are stimulated as possible, thereby making it more likely that a protective immune response will be generated.

It is thought that such an approach will be most useful in generating an immune response against at least two antigens wherein the antigens not complexed to a protein of the invention are external pathogenic antigens (in other words, antigens substantially exposed on the outside of a pathogen and which are generally ‘visible’to the immune system), for example the HPV L1 and L2 proteins, the Hepatitis C E1 protein, influenza virus HA or NA proteins, the RSV F, G or SH proteins, the HBV HBs protein, the HIV gp120 protein, Dengue virus E protein, VZV gE protein, CMV gB protein and EBV gp350 protein, or immunogenic fragments thereof whilst the antigen or antigens complexed to a protein of the invention are internal pathogenic antigens. Examples of the latter include the HPV E1, E2, E3, E4, E5, E6, E7, E8, E9 antigens, the HCV NS1, NS2, NS3, NS4a, 4b, NS5a, 5b proteins, the influenza virus matrix, nucleoprotein, NS1, NS2, PB1, PB2 or PA proteins, RSV M1, M2-1, M2-2, L, NS1, NS2, P protein or nucleoprotein, Hepatitis B virus HB core protein, HIV Nef, tat, P27, F4 or P24 protein, CMV pp 65 protein or Epstein Bar Virus latency related gene, or immunogenic fragments thereof. In one embodiment of the invention the antigens are from two different pathogens, whilst in another embodiment of the invention the antigens are from the same pathogen. In this embodiment one advantage provided by the invention is the provision of CD8 and CD4 responses to the same antigen.

In one embodiment of the invention there are only two antigens present, one of which is not complexed with the protein of the invention, and one of which is complexed with a protein of the invention. In a further embodiment, there is only one antigen complexed to the protein of the invention, but the composition comprises more than one antigen which is not complexed to a protein of the invention. In a further embodiment, there are one or more antigens present which are not complexed to a protein of the invention, and more than one antigen present which is complexed to a protein of the invention. In this embodiment, each complexed antigen may be complexed to a separate protein of the invention, or more than one antigen for example 2, 3, 4 or 5 antigens may be complexed to one protein of the invention.

In a further embodiment, the composition may comprise, as well as a protein of the invention, a further protein as described in patent application WO05112991. In this embodiment, the B subunit of Shiga toxin or an immunologically functional equivalent thereof which is able to bind the Gb3 receptor is also used to complex an antigen. Thus, for example, a composition of the present invention may comprise one or more free antigens, one or more antigens complexed with one or more proteins of the invention, and one or more antigens complexed with the B subunit of Shiga toxin or an immunologically functional equivalent therefore as described in WO2005/112991.

In one embodiment, the antigens are viral antigens. In one aspect suitable viral antigens for use either in complexing to the protein of the invention, or for use in an uncomplexed form, may be selected from the lists given above.

In one aspect the antigen not complexed to a protein of the invention is the HPV L1 protein or immunogenic fragment thereof. Suitable L1 proteins and L1 protein fragments are well known in the art, for example as disclosed in WO2004/056389 and references therein, all herein incorporated by reference. In one aspect the L1 protein is full length L1. In one aspect the L1 protein is a truncated L1 protein. In one aspect the L1 protein is in the form of a virus like particle (VLP), the VLP being made up of either full length or truncated L1. Where L1 is truncated, then in one aspect the truncation removes a nuclear localisation signal. In one aspect the truncation is a C terminal truncation. In one aspect the C terminal truncation removes less than 50 amino acids, for example less than 40 amino acids. Where the L1 is an HPV 16 VLP then in one aspect the C terminal truncation removes 34 amino acids from HPV 16 L1. Where the VLP is an HPV 18 VLP then in one aspect the C terminal truncation removes 35 amino acids from HPV 18 L1. L1 may be selected from any suitable HPV, for example oncogenic HPV types such as HPV 16, 18, 31, 33, 35, 39, 45, 51, 52, 56, 58, 59, 66, 68.

Truncated L1 proteins are suitably functional L1 protein derivatives. Functional L1 protein derivatives are capable of raising an immune response (if necessary, when suitably adjuvanted), said immune response being capable of recognising a VLP consisting of the full length L1 protein and/or the HPV type from which the L1 protein was derived.

Where one antigen not complexed to a protein of the invention is the HPV L1 protein or immunogenic fragment thereof, then in one aspect one antigen complexed to a protein of the invention is the HPV E2 protein, or E4 protein, or E5 protein, or E6 protein, or E7 protein, or immunogenic fragments thereof.

In one embodiment of the invention, the composition of the invention comprises HPV 16 L1 and HPV 18 L1 as free antigens, and one or more HPV early proteins as complexed antigen. Preferably early proteins are present from both HPV 16 and 18. Preferably more than one early protein is present. In one aspect of this embodiment, the composition comprises HPV16 E7 and HPV 18 E7. In a further particular aspect of this embodiment, the composition comprises HPV16 E2, HPV 18E2, HPV 16 E6 and HPV 18 E6 as complexed antigens. In one aspect of this embodiment, HPV 16 and HPV 18 L1 are present in the form of VLPs.

In one aspect one antigen not complexed to a protein of the invention is the HCV E1 protein or immunogenic fragment thereof, such as a truncate thereof, for example, a C terminal E1 truncate, and one antigen complexed to a protein of the invention is the HCV NS3 protein or immunogenic fragment thereof.

In one aspect one antigen not complexed to a protein of the invention is the VZv gE protein or immunogenic fragment thereof. One antigen complexed to a protein of the invention in this case may be, for example, IE63 or IE62, or immunogenic fragments thereof.

In one aspect one antigen not complexed to a protein of the invention is the HCMV gB protein or immunogenic fragment thereof, or the gH protein or immunogenic fragment thereof. In one aspect one antigen not complexed to a protein of the invention is the pp 65 protein or immunogenic fragment thereof, or the major immediate early protein IE1 72, or immunogenic fragment thereof.

In one aspect of the invention, one antigen not complexed to a protein of the invention is an influenza subunit antigen, for example NA or HA or immunogenic fragment thereof or combinations thereof. In a further aspect, an influenza split preparation may be used in the composition to provide the antigens not complexed to a protein of the invention. One antigen complexed to a protein of the invention in these cases may be, for example, influenza virus matrix protein, NP, PB1, PB2, PA, NS2 or NS1 protein or immunogenic fragments thereof.

In one aspect of the invention, one antigen not complexed to a protein of the invention is an RSV F, G or SH protein or immunogenic fragment thereof. In this case, one antigen complexed to a protein of the invention may be, for example, an RSV M1, M2-1, M2-2, L, P, NS1, NS2, N protein or an immunogenic fragment thereof.

In one aspect of the invention, one antigen not complexed to a protein of the invention is an HBV HBs protein or an immunogenic fragment thereof. In this case, one antigen complexed to a protein of the invention may be, for example, HB core protein or an immunogenic fragment thereof.

In one aspect of the invention, one antigen not complexed to a protein of the invention is an HIV gp120 protein or an immunogenic fragment thereof. In this case, one antigen complexed to a protein of the invention may be, for example, an HIV Nef, tat, P27, F4 or P24 protein or an immunogenic fragment thereof.

In one aspect of the invention, one antigen not complexed to a protein of the invention is a Dengue virus E protein or an immunogenic fragment thereof. In this case, one antigen complexed to a protein of the invention may be, for example, a dengue virus NS1 protein or an immunogenic fragment thereof.

In one aspect of the invention, one antigen not complexed to a protein of the invention is an EBV gp350 protein or an immunogenic fragment thereof. In this case, one antigen complexed to a protein of the invention may be, for example, an EBV latency related gene product or an immunogenic fragment thereof.

Example of immunogenic fragments of antigens include, for example, peptides comprising B and/or T cell epitopes, and which can be used to stimulate an immune response.

Where 2 different antigens are used from the same virus, such as HPV L1 and HPV E5, then in one aspect the antigens are from the same viral type or subtype—e.g. both from HPV 16. This principle can be applied to antigen combinations from other viruses.

In a further aspect of the invention, the vaccine compositions of the invention comprise an antigen complexed to a protein of the invention, and further comprise the same antigen as free antigen, i.e. not complexed to a protein of the invention

In both of the above described aspects of the invention, the vaccine composition further comprises an adjuvant as described herein.

The amount of antigen in each vaccine dose is selected as an amount which induces an immunoprotective response without significant, adverse side effects in typical vaccines. Such amount will vary depending upon which specific immunogen is employed and how it is presented.

Generally, it is expected that each human dose will comprise 0.1-1000 μg of antigen, preferably 0.1-500 μg, preferably 0.1-100 μg, most preferably 0.1 to 50 μg. An optimal amount for a particular vaccine can be ascertained by standard studies involving observation of appropriate immune responses in vaccinated subjects. Following an initial vaccination, subjects may receive one or several booster immunisation adequately spaced. Such a vaccine formulation may be applied to a mucosal surface of a mammal in either a priming or boosting vaccination regime; or alternatively be administered systemically, for example via the transdermal, subcutaneous or intramuscular routes. Intramuscular administration is preferred.

The amount of 3 D MPL used is generally small, but depending on the vaccine formulation may be in the region of 1-1000 μg per dose, preferably 1-500 μg per dose, and more preferably between 1 to 100 μg per dose.

The amount of CpG or immunostimulatory oligonucleotides in the adjuvants or vaccines of the present invention is generally small, but depending on the vaccine formulation may be in the region of 1-1000 μg per dose, preferably 1-500 μg per dose, and more preferably between 1 to 100 μg per dose.

The amount of saponin for use in the compositions of the present invention may be in the region of 1-1000 μg per dose, preferably 1-500 μg per dose, more preferably 1-250 μg per dose, and most preferably between 1 to 100 μg per dose.

The formulations of the present invention may be used for both prophylactic and therapeutic purposes. Accordingly the invention provides a vaccine composition as described herein for use in medicine.

In a further embodiment there is provided a method of treatment of an individual susceptible to or suffering from a disease by the administration of a composition as substantially described herein.

Also provided is a method to prevent an individual from contracting a disease selected from the group comprising infectious bacterial and viral diseases, parasitic diseases, particularly intracellular pathogenic disease, proliferative diseases such as prostate, breast, colorectal, lung, pancreatic, renal, ovarian or melanoma cancers; non-cancer chronic disorders, allergy comprising the administration of a composition as substantially described herein to said individual.

Furthermore, there is described a method of inducing a CD8+ antigen specific immune response in a mammal, comprising administering to said mammal a composition of the invention. Further there is provided a method of manufacture of a vaccine comprising admixing an antigen in combination with a non-live vector or immunological functional equivalent thereof with an adjuvant.

Examples of suitable pharmaceutically acceptable excipients for use in the combinations of the present invention include, among others, water, phosphate buffered saline, isotonic buffer solutions

The present invention is exemplified by reference to the following examples and Figures. In all figures, adeno-ova (adenovirus vector containing OVA protein) was used as a positive control in first injection. P/B (prime/boost) is a positive control with first injection of Adeno-Ova, and second, boost injection of Ova in AS A.

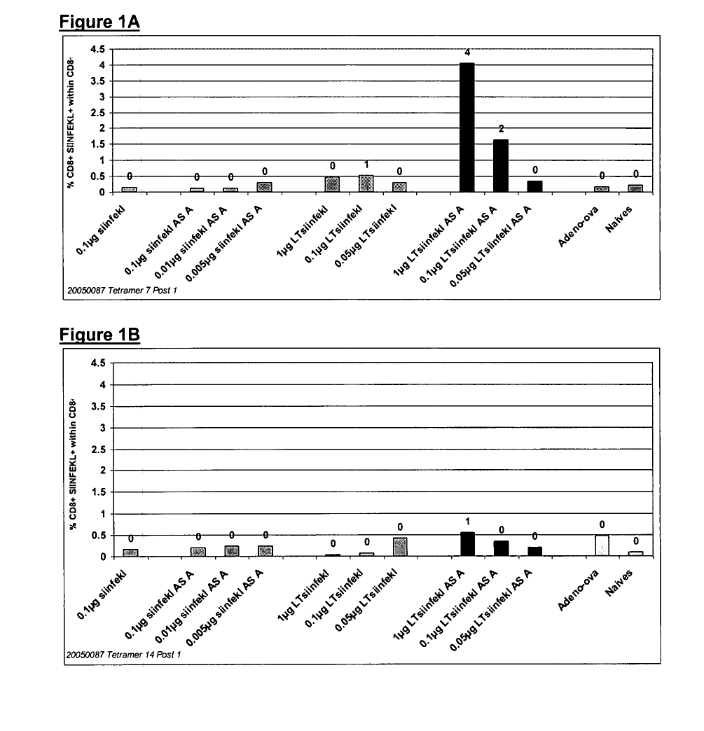

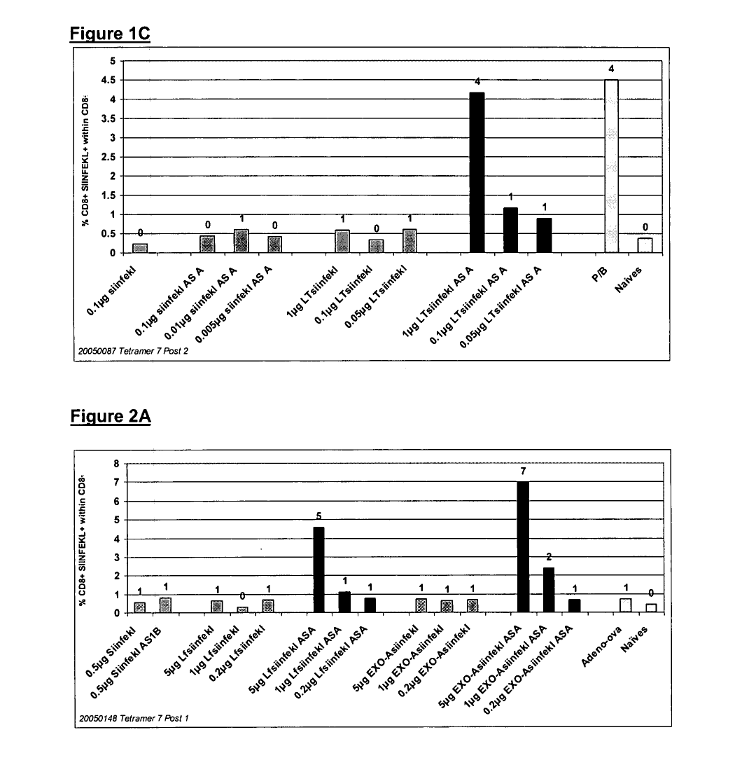

FIG. 1A: Siinfekl-specific CD 8 frequency in PBLs 7 days after primary injection with AS A LTx-Siinfekl vaccine

FIG. 1B: Siinfekl-specific CD 8 frequency in PBLs 15 days after primary injection with AS A LTx-Siinfekl vaccine

FIG. 1C: -Siinfekl-specific CD8 frequency in PBLs 7 days after second injection with AS A LTx-Siinfekl vaccine

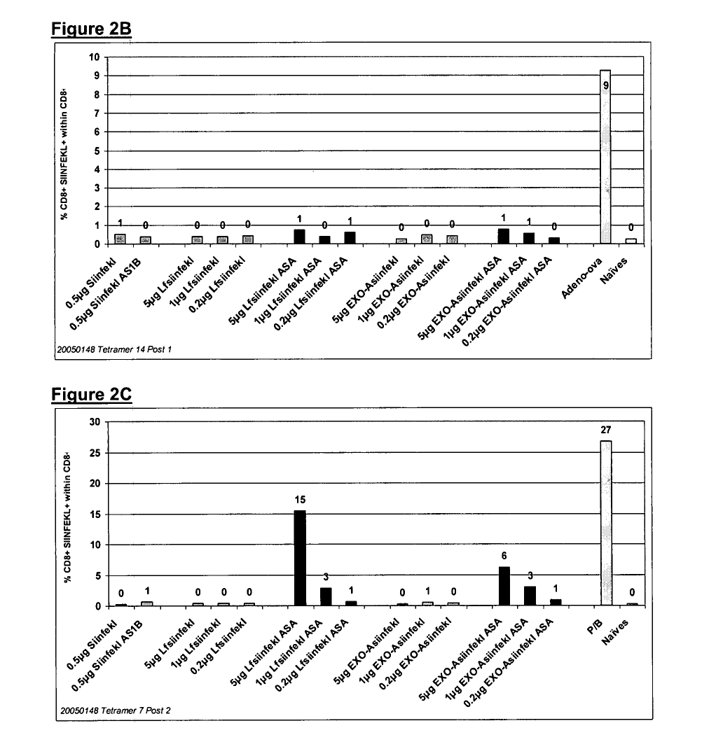

FIG. 2A: Siinfekl-specific CD 8 frequency in PBLs 7 days after primary injection with Exo-A-Siinfekl AS A or LF-Siinfekl AS A vaccine

FIG. 2B: Siinfekl-specific CD 8 frequency in PBLs 14 days after primary injection with Exo-A-Siinfekl AS A or LF-Siinfekl AS A vaccine

FIG. 2C: -Siinfekl-specific CD8 frequency in PBLs 7 days after second injection with Exo-A-Siinfekl AS A or LF-Siinfekl AS A vaccine

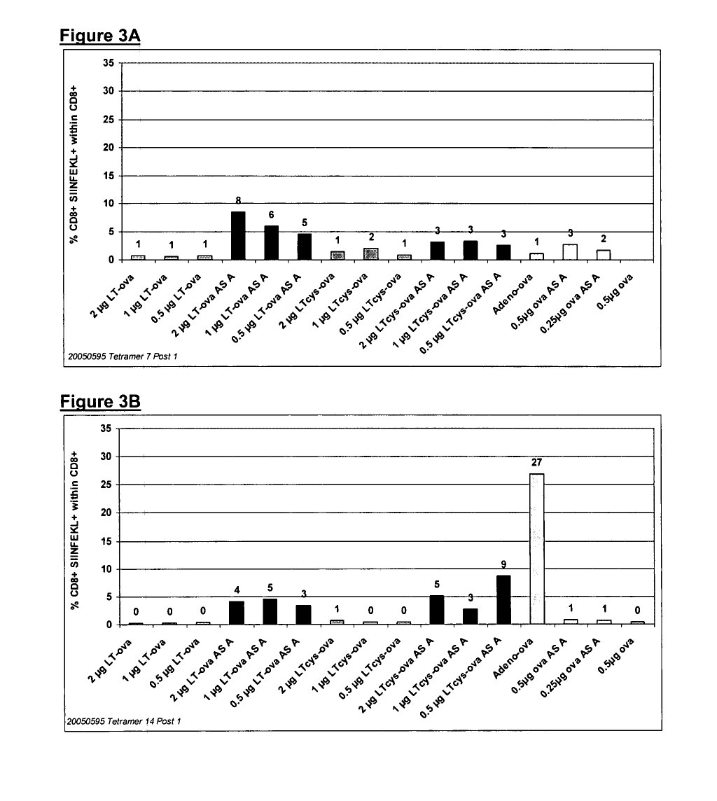

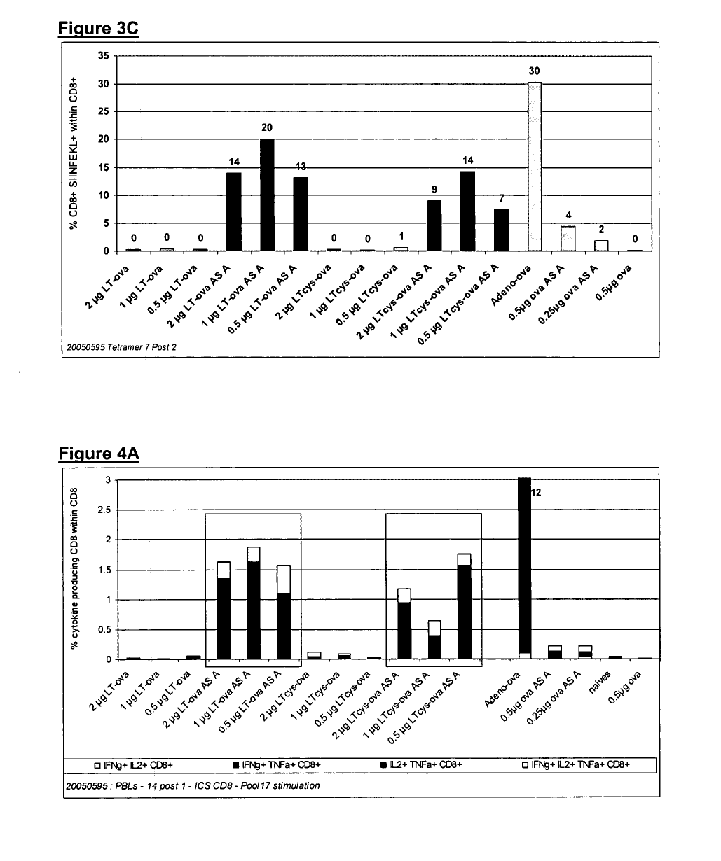

FIG. 3A: Siinfekl-specific CD 8 frequency in PBLs 7 days after primary injection with LT-Ova or LT-cys-Ova with or without AS A

FIG. 3B: Siinfekl-specific CD 8 frequency in PBLs 14 days after primary injection with LT-Ova or LT-Cys-ova with or without AS A

FIG. 3C: Siinfekl-specific CD 8 frequency in PBLs 7 days after second injection with LT-Ova or LT-cys-ova with our without AS A

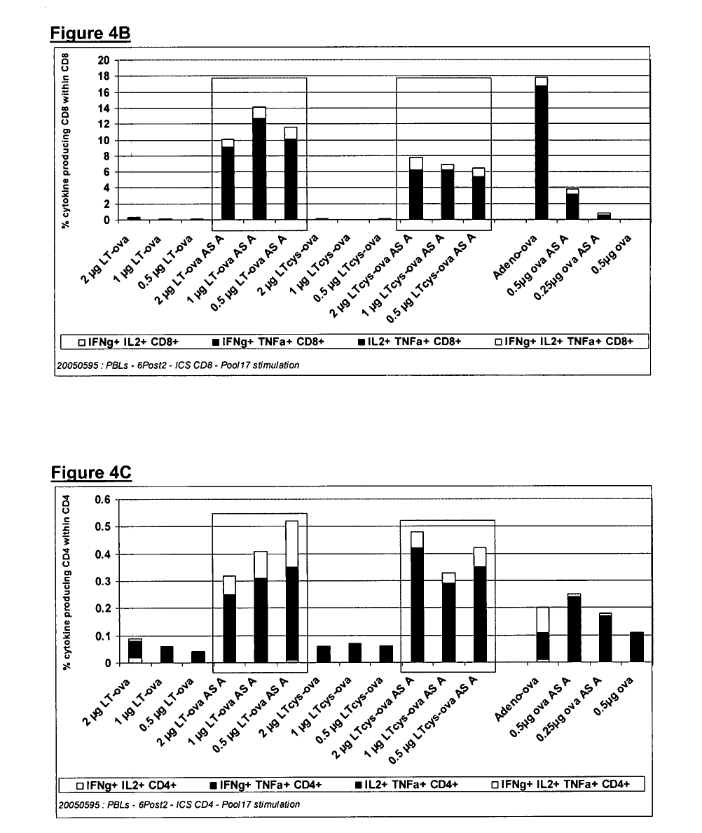

FIG. 4A: cytokine producing CD8+ T cells frequency (%) 14 days following primary injection with LT-Ova or LT-cys Ova with or without AS A. Graphs are showing CD8 producer of at least two cytokines (IFNg, IL2, TNFa).

FIG. 4B: cytokine producing CD8+ T cells frequency (%) 6 days following second injection with LT-Ova or LT-cys Ova with or without AS A. Graphs are showing CD8 producer of at least two cytokine (IFNg, IL2, TNFa).

FIG. 4C: cytokine producing CD4+ T cells frequency (%) 6 days following second injection with LT-Ova or LT-cys Ova with or without AS A. Graphs are showing CD4 producer of at least two cytokines (IFNg, IL2, TNFa).

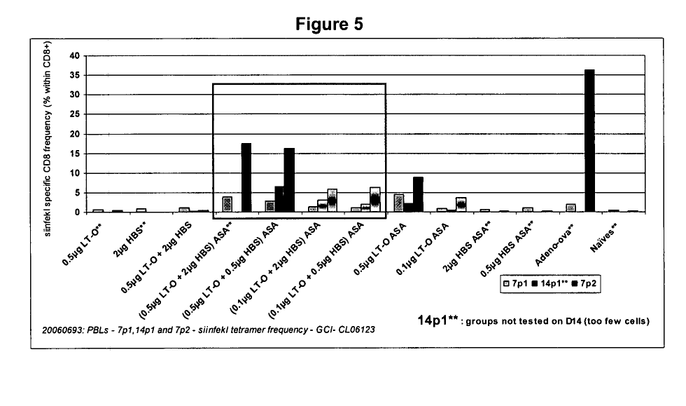

FIG. 5: Siinfekl-specific CD8 frequency in PBLs following injection with ASA adjuvanted composition comprising LT-Ova and HBs as free antigen, showing tetramer responses against Ova 7 post 1, 14 post 1 and 7 post 2.

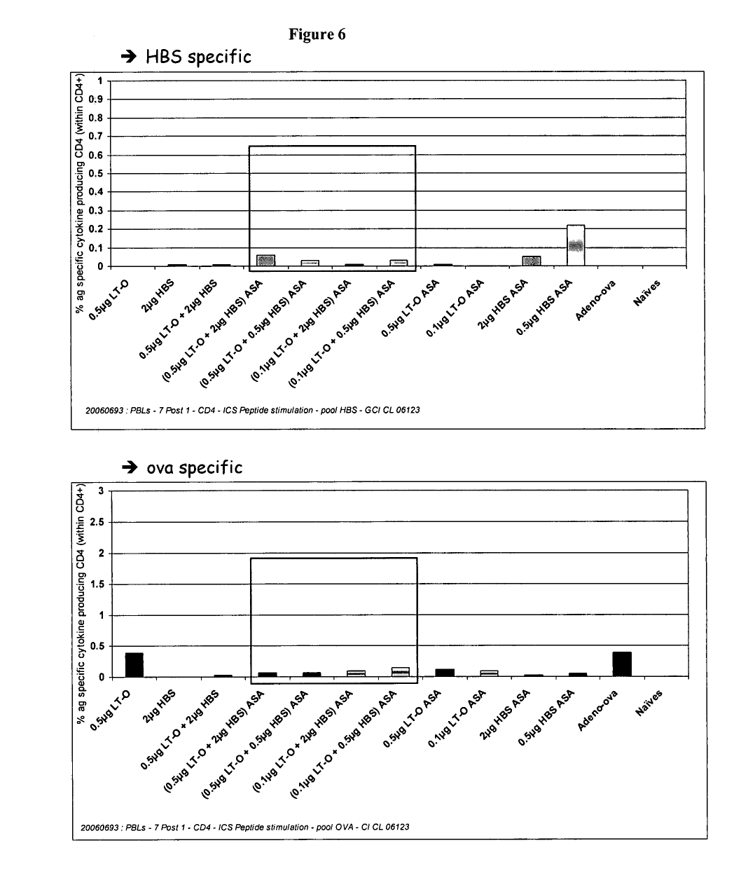

FIG. 6: % antigen specific cytokine producing CD4 frequency in PBLs 7 days post 1^{st} injection with ASA adjuvanted composition comprising LT-Ova and HBs as free antigen, top graph showing HBs responses, bottom graph showing Ova responses.

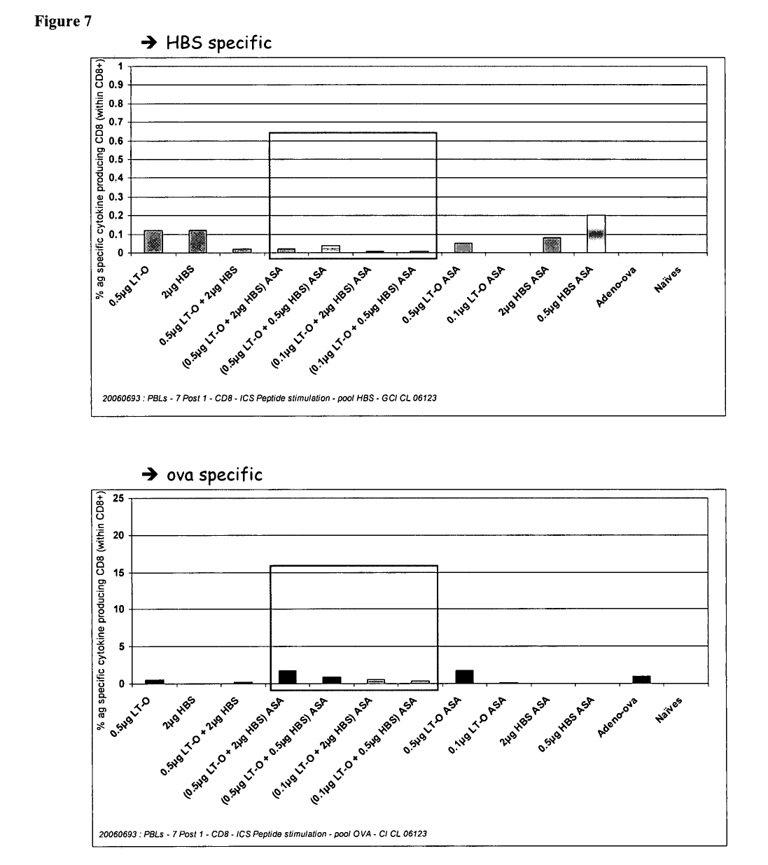

FIG. 7: % antigen specific cytokine producing CD8 frequency in PBLs 7 days post 1^{st} injection with ASA adjuvanted composition comprising LT-Ova and HBs as free antigen, top graph showing HBs responses, bottom graph showing Ova responses.

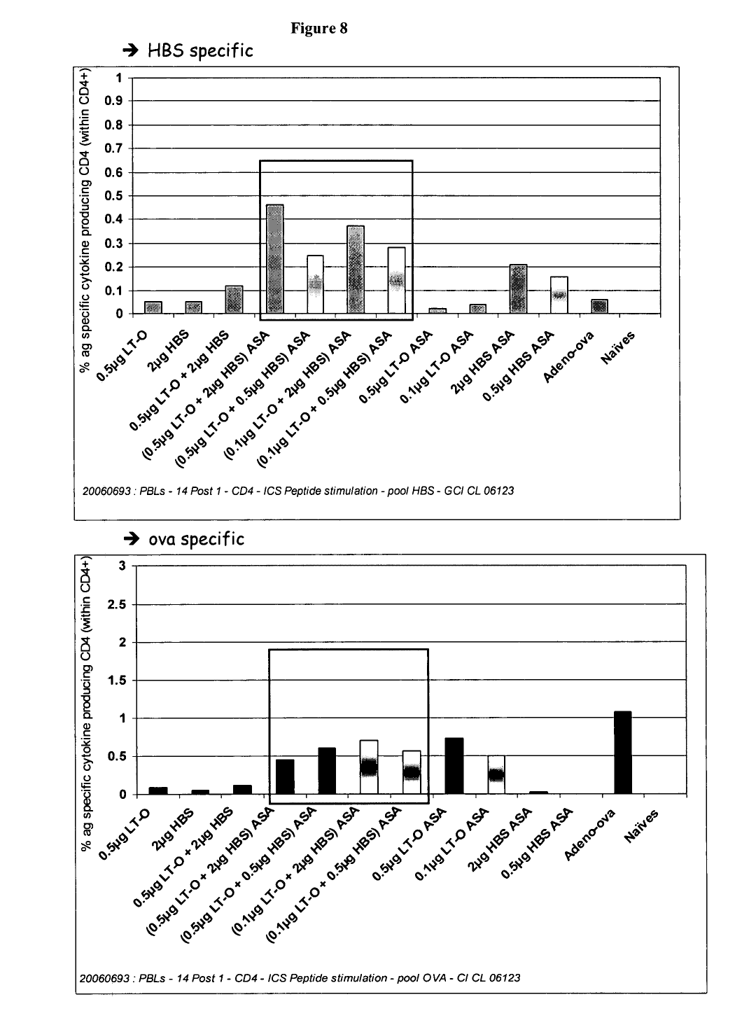

FIG. 8: % antigen specific cytokine producing CD4 frequency in PBLs 14 days post 1^{st} injection with ASA adjuvanted composition comprising LT-Ova and HBs as free antigen, top graph showing HBs responses, bottom graph showing Ova responses.

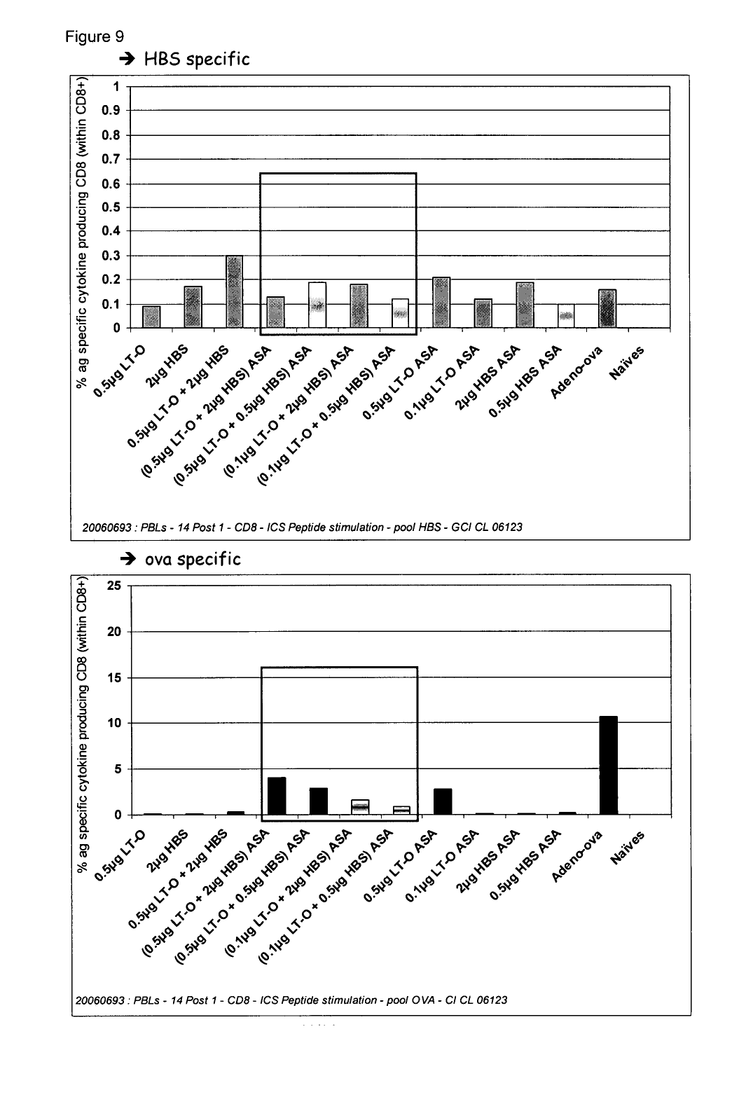

FIG. 9: % antigen specific cytokine producing CD8 frequency in PBLs 14 days post 1^{st} injection with ASA adjuvanted composition comprising STx-Ova and HBs as free antigen, top graph showing HBs responses, bottom graph showing Ova responses.

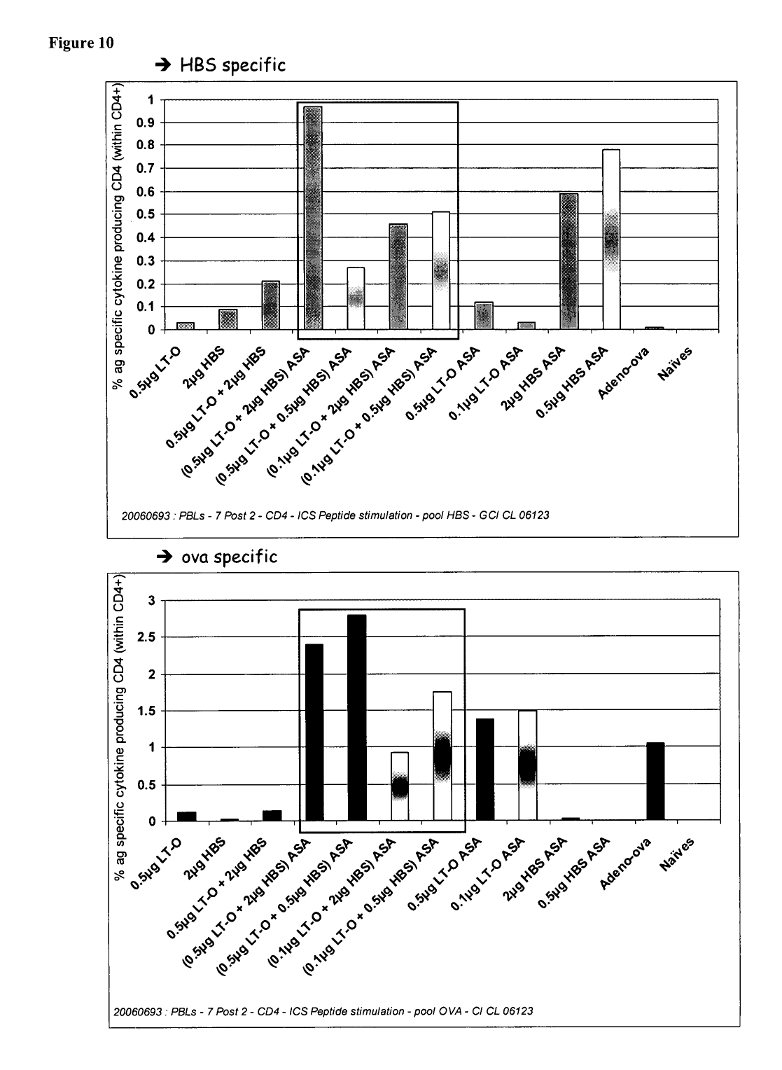

FIG. 10: % antigen specific cytokine producing CD4 frequency in PBLs 7 days post 2^{nd} injection with ASA adjuvanted composition comprising LT-Ova and HBs as free antigen, top graph showing HBs responses, bottom graph showing Ova responses.

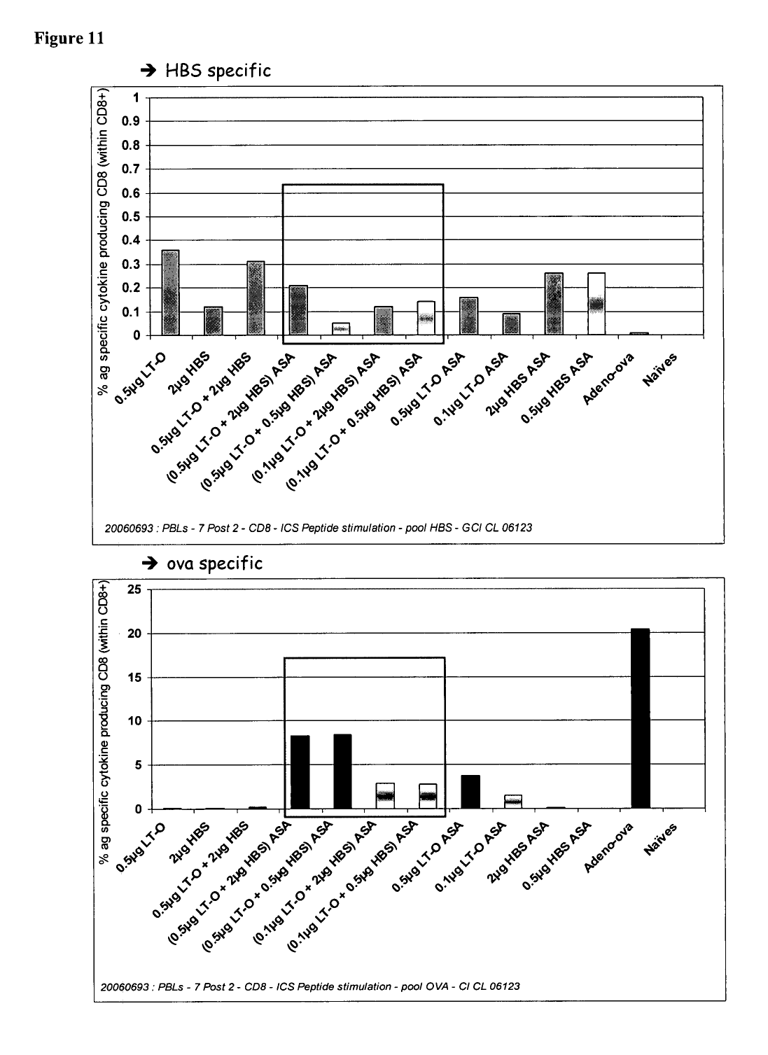

FIG. 11: % antigen specific cytokine producing CD8 frequency in PBLs 7 days post 2^{nd} injection with ASA adjuvanted composition comprising LT-Ova and HBs as free antigen, top graph showing HBs responses, bottom graph showing Ova responses.

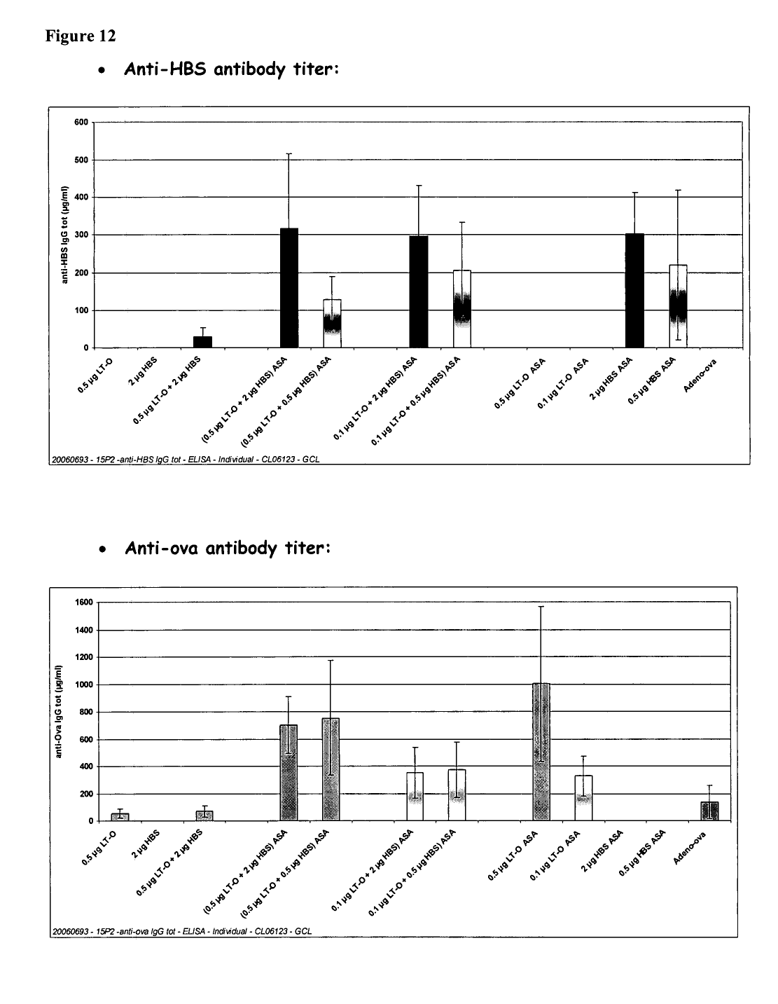

FIG. 12: Antibody responses against HBs (top) and Ova (bottom) 14 days post 2^{nd} injection with an ASA adjuvanted composition containing LT-Ova and HBs as free antigen.

### EXAMPLES

### Example 1

### Reagents and Media

### 1.1 Preparation of LTB, LTB-Cys and LTB-Siinfekl Recombinants

The LTB, LTB-cys (SEQ ID NO.7) and LTB-siinfekl (SEQ ID NO. 8) coding sequences were amplified by PCR and cloned into pET expression vectors for expression in E Coli. A total protein extract was obtained from a bacterial pellet at OD₍₆₂₀₎ 60 using the French press. After 30′ centrifugation at 15000 g, the supernatant was harvested and precipitated by adding (NH4)₂SO4 (4.95 g/10 ml) and incubating at least 4 hours at 4° C. The protein pellet was harvested after centrifugation, dissolved in PBS (4 times concentration), and dialyzed intensively against the same buffer. The insoluble fraction was eliminated by centrifugation and 0.22 μm filtration. The clarified supernatant was loaded on a XK16/15 cm length column containing 15 ml PBS pre-equilibrated immobilized D-galactose resin (Calbiochem), and washed with PBS until the optical density dropped to basal level. The bound protein was eluted with 1M galactose in PBS. After dialysis, the recovered protein is visualized by SDS Page, Coomassie staining and Western blotting. This method of purifying proteins using a D-galactose resin may also be used to determine whether a protein of interest binds the GM1 receptor.

SEQ ID NO. 7

ATGAATAAAGTAAAATGTTATGTTTTATTTACGGCGTTACTATCCTCTCT
ATGTGCATACGGAGCTCCCCAGTCTATTACAGAACTATGTTCGGAATATC
GCAACACACAAATATATACGATAAATGACAAGATACTATCATATACGGAA
TCGATGGCAGGCAAAAGAGAAATGGTTATCATTACATTTAAGAGCGGCGC
AACATTTCAGGTCGAAGTCCCGGGCAGTCAACATATAGACTCCCAAAAAA
AAGCCATTGAAAGGATGAAGGACACATTAAGAATCACATATCTGACCGAG
ACCAAAATTGATAAATTATGTGTATGGAATAATAAAACCCCCAATTCAAT
TGCGGCAATCAGTATGGAAAACTGCTAA

SEQ ID NO. 8

ATGAATAAAGTAAAATGTTATGTTTTATTTACGGCGTTACTATCCTCTCT
ATGTGCATACGGAGCTCCCCAGTCTATTACAGAACTATGTTCGGAATATC
GCAACACACAAATATATACGATAAATGACAAGATACTATCATATACGGAA
TCGATGGCAGGCAAAAGAGAAATGGTTATCATTACATTTAAGAGCGGCGC
AACATTTCAGGTCGAAGTCCCGGGCAGTCAACATATAGACTCCCAAAAAA
AAGCCATTGAAAGGATGAAGGACACATTAAGAATCACATATCTGAGCGAG
ACCAAAATTGATAAATTATGTGTATGGAATAATAAAACCCCCAATTCAAT
TGCGGCAATCAGTATGGAAAACAGCCAGCTTGAGAGTATAATCAACTTTG
AAAAACTGACTGAATGGCGCGGCCGCTAG

The LTB and LTB-cys vector (SEQ ID NO. 7) were conjugated to the commercially available full-length chicken Ovalbumin antigen as described in the following sections.

The LTB-siinfekl (SEQ ID NO. 8) recombinant was directly formulated in adjuvant system A noted below.

### Preparation of LTB/OVA Conjugate

The commercially available full-length chicken Ovalbumin antigen (5 mg) was reduced to expose SH groups by DTT treatment for 2 hours at room temperature. DTT was removed using a PD10 (Sephadex G-25, Amersham) column (elution with 2 mM phosphate buffer pH 6.8, fractions of 1 ml). The LTB vector described above (8 mg) was activated using a 10-fold molar excess of SGMBS for 1 hour at room temperature. The excess of SGMBS was removed using a PD10 column (elution with 100 mM phosphate buffer pH 7.2, fractions of 1 ml). For conjugation, equimolar amounts of reduced Ovalbumin (OVA-SH) and activated LTB were reacted for 1 hour at room temperature. The resulting conjugate was purified by molecular filtration on a S-300 HR Sephacryl column (elution with 100 mM Phosphate buffer pH 6.8, fractions of 1 ml).

The LTB/OVA conjugate was then formulated in adjuvant system A noted below.

### Preparation of LTB-Cys/OVA Conjugate

The commercially available full-length chicken Ovalbumin antigen (10 mg) was activated using a 80-fold molar excess of SGMBS for 1 hour at room temperature. The excess of SGMBS was removed using a PD10 column (elution with 1 ml fractions of DPBS buffer (NaCl 136.87 mM, KCl 2.68 mM, Na2HPO4 8.03 mM, KH2PO4 1.47 mM pH 7.5), fractions of 1 ml).

For conjugation, equimolar amounts of LTB-cys and activated Ovalbumin were reacted for 1 hour at room temperature. The resulting conjugate was purified by molecular filtration on a S-300 HR Sephacryl column (elution with DPBS buffer, fractions of 4 ml).

The LTB-cys/OVA conjugate was then formulated in adjuvant system A noted below.

### 1.2 Preparation of LFn-Siinfekl and LFn-OVA¹⁶¹⁻¹⁹¹ Recombinants

Two synthetic genes were prepared that contained the amino terminal 255 amino acids from Anthrax LF toxin flanked by a 6×His tail and either the Siinfekl coding sequence or a larger Ovalbumin fragment containing this epitope (fragment 161-291) (SEQ ID No. 9 and 10, respectively). The resulting products were cloned into a pET expression vector for expression in E Coli. Cells were recovered by centrifugation, concentrated (25 to 40×) and lysed using a French press. Aggregates were dissociated in 6 M urea overnight at 4° C. To purify the recombinant proteins, 5 ml of previously equilibrated Ni-NTA resin (Qiagen) were added to the lysate, incubated 2 hours at 4° C. on a rotating wheel and loaded onto a polyprep disposable column (BioRad). The column was washed three times with 15 ml of a 300 mM NaCl, 6 M urea, 5 mM imidazole, 50 mM Phosphate buffer pH8 before elution with 4×2 ml of the same buffer containing 500 mM imidazole The recovered proteins were visualized by SDS Page, Coomassie staining and Western blotting, and urea was removed by dialysis.

SEQ ID No. 9

| | |
|---|---|
| ATGGGCCACCATCACCATCACCATTCTTCTGGTGCGGGCG | 40 |
| GTCATGGTGATGTAGGTATGCACGTAAAAGAGAAAGAGAA | 80 |
| AAATAAAGATGAGAATAAGAGAAAAGATGAAGAACGAAAT | 120 |
| AAAACACAGGAAGAGCATTTAAAGGAAATCATGAAACACA | 160 |
| TTGTAAAAATAGAAGTAAAAGGGGAGGAAGCTGTTAAAAA | 200 |
| AGAGGCAGCAGAAAAGCTACTTGAGAAAGTACCATCTGAT | 240 |
| GTTTTAGAGATGTATAAAGCAATTGGAGGAAAGATATATA | 280 |
| TTGTGGATGGTGATATTACAAAACATATATCTTTAGAAGC | 320 |
| ATTATCTGAAGATAAGAAAAAAATAAAAGACATTTATGGG | 360 |
| AAAGATGCTTTATTACATGAACATTATGTATATGCAAAAG | 400 |
| AAGGATATGAACCCGTACTTGTAATCCAATCTTCGGAAGA | 440 |
| TTATGTAGAAAATACTGAAAAGGCACTGAACGTTTATTAT | 480 |
| GAAATAGGTAAGATATTATCAAGGGATATTTTAAGTAAAA | 520 |
| TTAATCAACCATATCAGAAATTTTTAGATGTATTAAATAC | 560 |
| CATTAAAAATGCATCTGATTCAGATGGACAAGATCTTTTA | 600 |
| TTTACTAATCAGCTTAAGGAACATCCCACAGACTTTTCTG | 640 |
| TAGAGTTCTTGGAACAAAATAGCAATGAGGTACAAGAAGT | 680 |
| ATTTGCGAAAGCTTTTGCATATTATATCGAGCCACAGCAT | 720 |
| CGTGATGTTTTACAGCTTTATGCACCGGAAGCTTTTAATT | 760 |
| ACATGGATAAATTTAACGAACAAGAAATAAATCTATCCGG | 800 |
| ATCCCAGCTTGAGAGTATAATCAACTTTGAAAAACTGACT | 840 |
| GAATGGTGA | 849 |

SEQ ID No. 10

| | |
|---|---|
| ATGGGCCACCATCACCATCACCATTCTTCTGGTGCGGGCG | 40 |
| GTCATGGTGATGTAGGTATGCACGTAAAAGAGAAAGAGAA | 80 |
| AAATAAAGATGAGAATAAGAGAAAAGATGAAGAACGAAAT | 120 |
| AAAACACAGGAAGAGCATTTAAAGGAAATCATGAAACACA | 160 |
| TTGTAAAAATAGAAGTAAAAGGGGAGGAAGCTGTTAAAAA | 200 |
| AGAGGCAGCAGAAAAGCTACTTGAGAAAGTACCATCTGAT | 240 |
| GTTTTAGAGATGTATAAAGCAATTGGAGGAAAGATATATA | 280 |
| TTGTGGATGGTGATATTACAAAACATATATCTTTAGAAGC | 320 |
| ATTATCTGAAGATAAGAAAAAAATAAAAGACATTTATGGG | 360 |
| AAAGATGCTTTATTACATGAACATTATGTATATGCAAAAG | 400 |
| AAGGATATGAACCCGTACTTGTAATCCAATCTTCGGAAGA | 440 |
| TTATGTAGAAAATACTGAAAAGGCACTGAAGGTTTATTAT | 480 |
| GAAATAGGTAAGATATTATCAAGGGATATTTTAAGTAAAA | 520 |
| TTAATCAACCATATCAGAAATTTTTAGATGTATTAAATAC | 560 |
| CATTAAAAATGCATCTGATTCAGATGGACAAGATCTTTTA | 600 |
| TTTACTAATCAGCTTAAGGAACATCCCACAGACTTTTCTG | 640 |
| TAGAGTTCTTGGAACAAAATAGCAATGAGGTACAAGAAGT | 680 |
| ATTTGCGAAAGCTTTTGCATATTATATCGAGCCACAGCAT | 720 |
| CGTGATGTTTTACAGCTTTATGCACCGGAAGCTTTTAATT | 760 |
| ACATGGATAAATTTAACGAACAAGAAATAAATCTATCCGG | 800 |
| ATCCGTCCTTCAGCCAAGCTCCGTGGATTCTCAAACTGCA | 840 |
| ATGGTTCTGGTTAATGGCATTGTCTTCAAAGGACTGTGGG | 880 |
| AGAAAACATTTAAGGATGAAGACACACAAGCAATGCCTTT | 920 |
| GAGAGTGACTGAGCAAGAAAGCAAACCTGTGCAGATGATG | 960 |
| TACCAGATTGGTTTATTTAGAGTGGCATCAATGGCTTCTG | 1000 |
| AGAAAATGAAGATCCTGGAGCTTCCATTTGCCAGTGGGAC | 1040 |
| AATGAGCATGTTGGTGCTGTTGCCTGATGAAGTCTCAGGC | 1080 |
| CTTGAGCAGCTTGAGAGTATAATCAACTTTGAAAAACTGA | 1120 |
| CTGAATGGACCAGTTCTAATGTTATGGAAGAGAGGAAGAT | 1160 |
| CAAAGTGTACTTACCTCGCATGAAGATGGAGGAAAAATGA | 120 |

The LFn-siinfekl (SEQ ID No. 9) and LFn-OVA¹⁶¹⁻¹⁹¹ (SEQ ID No. 10) recombinants were then formulated in adjuvant system A noted below.

### 1.3 Preparation Of ExoA-Siinfekl Recombinant

A synthetic gene was prepared (Seq ID No. 11—see below) that corresponds to a non toxic form of the Exotoxin A (deletion of E553), in which the Siinfekl epitope is introduced so that it replaces most of the Ib domain the toxin. The resulting product was cloned into a pET expression vector and expressed in E Coli. The recombinant protein was then extracted from inclusion bodies and purified essentially as described in FitzGerald et al, J Biol Chem 273, 9951, 1998.

SEQ ID NO. 11

ATGGCCGAGGAAGCCTTCGACCTCTGGAACGAATGCGCCAAAGCGTGCGT
GCTCGACCTCAAGGACGGCGTGCGTTCCAGCCGCATGAGCGTCGACCCGG
CCATCGCGGACACCAACGGGCAGGGCGTGCTGCACTACTCCATGGTCCTG
GAGGGCGGCAACGACGCGCTCAAGCTGGGCATCGACAACGCCCTCAGCAT
CACCAGCGACGGCCTGACCATCCGCCTCGAAGGCGGCGTCGAGCCGAACA
AGGCGGTGCGCTACAGCTACACGCGCCAGGCGCGCGGCAGTTGGTCGGTG
AACTGGCTGGTACCGATCGGCCACGAGAAGCCCTCGAACATCAAGGTGTT
CATCCACGAACTGAACGCCGGCAACCAGCTCAGCCACATGTCGCCGATCT
ACACCATCGAGATGGGCGACGAGTTGCTGGCGAAGCTGGCGCGCGATGCC
ACCTTCTTCGTCAGGGCGCACGAGAGCAACGAGATGCAGCCGACGCTCGC
CATCAGCCATGCCGGGGTCAGCGTGGTCATGGCCCAGACCCAGCCGCGCC
GGGAAAAGCGCTGGAGCGAATGGGCCAGCGGCAAGGTGTTGTGCCTGCTC
GACCCGCTGGACGGGGTCTACAACTACCTCGCCCAGCAACGCTGCAACCT
CGACGATACCTGGGAAGGCAAGATCTACCGGGTGCTCGCCGGCAACCCGG
CGAAGCATGACCTGGACATCAAACCCACGGTCATCAGTCATCGCCTGCAC
TTTCCCGAGGGCGGCAGCCTGGCCGCGCTGACCGCGCACCAGGCTTGCCA
CCTGCCGCTGGAGACTTTCACCCGTCATCGCCAGCCGCGCGGCTGGGAAC
AACTGGAGCAGTGCGGCTATCCGGTGCAGCGGCTGGTCGCCCTCTACCTG
GCGGCGCGGCTGTCGTGGAACCAGGTCGACCAGGTGATCCGCAACGCCCT
GGCCAGCCCCGGCAGCGGCGGCGACCTGGGCGAAGCGATCCGCGAGCAGC
CGGAGCAGGCCCGTCTGGCCGTGACCCTGGCCGCCGCCGAGAGCGAGCGC
TTCGTCCGGCAGGGCACCGGCAACGACGAGGCCGGCGCGGCCAACCTGCA
CTGCCAGCTTGAGAGTATAATCAACTTTGAAAAACTGACTGAATGGTGCA
TGCAGGGCCCGGCGGACAGCGGCGACGCCCTGCTGGAGCGCAACTATCCC
ACTGGCGCGGAGTTCCTCGGCGACGGCGGCGACGTCAGCTTCAGCACCCG
CGGCACGCAGAACTGGACGGTGGAGCGGCTGCTCCAGGCGCACCGCCAAC
TGGAGGAGCGCGGCTATGTGTTCGTCGGCTACCACGGCACCTTCCTCGAA
GCGGCGCAAAGCATCGTCTTCGGCGGGGTGCGCGCGCGCAGCCAGGACCT
CGACGCGATCTGGCGCGGTTTCTATATCGCCGGCGATCCGGCGCTGGCCT
ACGGCTACGCCCAGGACCAGGAACCCGACGCACGCGGCCGGATCCGCAAC
GGTGCCCTGCTGCGGGTCTATGTGCCGCGCTCGAGCCTGCCGGGCTTCTA
CCGCACCAGCCTGACCCTGGCCGCGCCGGAGGCGGCGGGCGAGGTCGAAC
GGCTGATCGGCCATCCGCTGCCGCTGCGCCTGGACGCCATCACCGGCCCC
GAGGAGGAAGGCGGGCGCCTGACCATTCTCGGCTGGCCGCTGGCCGAGCG
CACCGTGGTGATTCCCTCGGCGATCCCCACCGACCCGCGCAACGTCGGCG
GCGACCTCGACCCGTCCAGCATCCCCGACAAGGAACAGGCGATCAGCGCC
CTGCCGGACTACGCCAGCCAGCCCGGCAAACCGCCGCGCGAGGACCTGAA
GTGA

The ExoA-siinfekl recombinant (SEQ ID No. 11) was then formulated in adjuvant system A noted below.

### 1.4 Galabiose Binding Assay

The Gb3 receptor preferentially recognized by the B subunit of Shiga toxin is a cell surface glycosphingolipid, globotriaosylceramide (Galα1-4Galβ1-4 glucosylceramide), where Gal is Galactose. The method described below is based on that described by Tarrago-Trani (Protein Extraction and Purification 38, pp 170-176, 2004), and involves an affinity chromatography on a commercially available galabiose-linked agarose gel (calbiochem). Galabiose (Galα1->4Gal) is the terminal carbohydrate portion of the oligosacharride moiety of Gb3 and is thought to represent the minimal structure recognized by the B subunit of Shiga toxin. This method has been successfully used to purify Shiga toxin directly from E. coli lysate. Therefore it can be assumed that proteins that bind this moiety will bind the Gb3 receptor.

The protein of interest in PBS buffer (500 μl) is mixed with 100 μl of immobilised galabiose resin (Calbiochem) previously equilibrated in the same buffer, and incubated for 30 min to 1 hour at 4° C. on a rotating wheel. After a first centrifugation at 5000 rpm for 1 min, the pellet is washed twice with PBS. The bound material is then eluated twice by re-suspending the final pellet in 2×500 μl of 100 mM glycine pH 2.5. Samples corresponding to the flow-through, the pooled washes and the pooled eluates are then analyzed by SDS Page, Coomassie staining and Western blotting. These analytical techniques allow identification of whether the protein is bound to the galabiose, and hence will bind the Gb3 receptor.

### 1.5 Preparation of Adjuvant System A: QS21 and 3D-MPL.

A mixture of lipid (such as phosphatidylcholine either from egg-yolk or synthetic) and cholesterol and 3 D-MPL in organic solvent, was dried down under vacuum (or alternatively under a stream of inert gas). An aqueous solution (such as phosphate buffered saline) was then added, and the vessel agitated until all the lipid was in suspension. This suspension was then microfluidised until the liposome size was reduced to about 100 nm, and then sterile filtered through a 0.2 μm filter. Extrusion or sonication could replace this step.

Typically the cholesterol:phosphatidylcholine ratio was 1:4 (w/w), and the aqueous solution was added to give a final cholesterol concentration of 5 to 50 mg/ml.

The liposomes have a defined size of 100 nm and are referred to as SUV (for small unilamelar vesicles). The liposomes by themselves are stable over time and have no fusogenic capacity. Sterile bulk of SUV was added to PBS to reach a final concentration of 10, 20 or 100 μg/ml of 3D-MPL. PBS composition was Na2HPO4: 9 mM; KH2PO4: 48 mM; NaCl: 100 mM pH 6.1. QS21 in aqueous solution was added to the SUV. This mixture is referred as DQMPLin. Siinfekl non-live vector was then added. Between each addition of component, the intermediate product was stirred for 5 minutes. The pH was checked and adjusted if necessary to 6.1+/−0.1 with NaOH or HCl.

In the experiments described in section 3.1 below, injection volume of 50 μl corresponded to 0.05, 0.1, 0.2 1 or 5 μg of Siinfekl recombinant non-live vector and 0.5 μg of both immunostimulants (3D-MPL and QS21).

### Example 2

### Vaccination of C57/B6 Mice with Vaccines of the Invention

### Example 2

### Vaccination of C57/B6 Mice with Vaccines of the Invention

Various formulations as described above were used to vaccinate 6-8 week old C57BL/B6 female mice (10/group). The mice received two injections spaced 14 days apart and were bled during weeks 1, 2, 3 and 4 (for actual bleed days see specific examples) The mice were vaccinated intramuscularly (injection into the left gastrocnemien muscle of a final volume of 50 μl). The ovalbumin recombinant adenovirus was injected at a dose of 5×10⁸ VP.

Ex vivo PBLs stimulation were performed in complete medium which is RPMI 1640 (Biowitaker) supplemented with 5% FCS (Harlan, Holland), 1 μg/ml of each anti-mouse antibodies CD49d and CD28 (BD, Biosciences), 2 mM L-glutamine, 1 mM sodium pyruvate, 10 μg/ml streptamycin sulfate, 10 units/ml penicillin G sodium (Gibco), 10 μg/ml streptamycin 50 μM B-ME mercaptoethanol and 100× diluted non-essential amino-acids, all these additives are from Gibco Life technologies. Peptide stimulations were always performed at 37° C., 5% CO2.

### 2.1 Immunological Assays:

### 2.1.1 Detection of Antigen-Specific T Cells

Isolation of PBLs and tetramer staining. Tetramer is available only for the ovalbumine antigen model (ova), the siinfekl-tetramer is commercially available (Immunomics Coulter). Blood was taken from retro-orbital vein (50 μl per mouse, 10 mice per group) and directly diluted in RPMI+heparin (LEO) medium. PBLs were isolated through a lymphoprep gradient (CEDERLANE). Cells were then washed, counted and finally 3×10⁵ cells were re-suspended in 50 μl FACS buffer (PBS, FCS1%, 0.002% NaN3) containing CD16/CD32 antibody (BD Biosciences) at 1/50 final concentration (f.c.). After 10 min., 50 μl of the tetramer mix was added to cell suspension. The tetramer mix contains 1 μl of siinfekl-H2 Kb tetramer-PE from Immunomics Coulter and anti-CD8a-PercP (1/100 f.c.) antibodies were added in the test. The cells were then left for 10 minutes at 37° C. before being washed once and analysed using a FACS Calibur™ with CELLQuest™ software, 3000 events within the gate of living CD8 are required per test.

### 2.1.2 Intracellular Cytokine Staining (ICS).

ICS was performed on blood samples taken as described in paragraph 2.1.1. This technology is applied for both antigen-models: ova and HBS.

10⁶ PBLs were re-suspended in complete medium supplemented with, when needed, either a pool of 15-mer HBS peptides (54 peptides covering the whole HBS sequence used at f.c. of 1 μg/ml of each peptide) or a pool of 17 15-mer Ova peptides (11 MHC classI-restricted peptides and 6 MHC classII-restricted peptides) present at a concentration of each 1 μg/ml. After 2 hours, 1 μg/ml Brefeldin-A (BD, Biosciences) was added for 16 hours and cells were collected after a total of 18 hours. Cells were washed once and then stained with anti-mouse antibodies all purchased at BD, Biosciences; all further steps were performed on ice. The cells were first incubated for 10 min. in 50 μl of CD16/32 solution (1/50 f.c., FACS buffer). 50 μl of T cell surface marker mix was added (1/100 CD8a perCp, 1/100 CD4 APCcy7) and the cells were incubated for 20 min. before being washed. Cells were fixed & permeabilized in 200 μl of perm/fix solution (BD, Biosciences), washed once in perm/wash buffer (BD, Biosciences) before being stained at 4° C. with anti IFNg-APC anti IL2-FITC and anti TNFa-PE either for 2 hours or overnight. Data were analysed using a FACS Calibur™ with CELLQuest™ software, 15000 events within the gate of living CD8 are required per test.

### 2.1.4 Ag Specific Antibody Titer (Individual Analysis of Total IgG): ELISA.

This technology is applied for both antigen-models: ova and HBS. Serological analysis was assessed 15 days. Mice (10 per group) were bled by retro-orbital puncture. Anti-HBS and Anti-ova total IgG were measured by ELISA. 96 well-plates (NUNC, Immunosorbant plates) were coated with antigen overnight at 4° C. (either 50 μl per well of HBS solution (HBS 10 μg/ml, PBS) or 50 μl per well of ova solution (ova 10 μg/ml, PBS). The plates were then washed in wash buffer (PBS/0.1% Tween 20 (Merck)) and saturated with 100 μl of saturation buffer (PBS/0.1% Tween 20/1% BSA/10% FCS) for 1 hour at 37° C. After 3 further washes in the wash buffer, 100 μl of diluted mouse serum was added and incubated for 90 minutes at 37° C. After another three washes, the plates were incubated for another hour at 37° C. with biotinylated anti-mouse total IgG diluted 1000 times in saturation buffer. After saturation 96 w plates were washed again as described above. A solution of streptavidin peroxydase (Amersham) diluted 1000 times in saturation buffer was added, 50 μl per well. The last wash was a 5 steps wash in wash buffer. Finally, 50 μl of TMB (3,3′,5,5′-tetramethylbenzidine in an acidic buffer—concentration of H₂O₂ is 0.01%—BIORAD) per well was added and the plates were kept in the dark at room temperature for 10 minutes

To stop the reaction, 50 μl of H₂SO₄ 0.4N was added per well. The absorbance was read at a wavelength of 450/630 nm by an Elisa plate reader from BIORAD. Results were calculated using the softmax-pro software.

### 3. Results

The results described below show that, using LFn, LT-B or Exo-A, the efficiency of a non-live vector system at inducing CD8 responses can be improved by combining it with adjuvant system A.

Evaluation of the Response with Adjuvant System A

The results shown in FIGS. 1 and 2 (methods carried out as in 2.1.1 above) show that, measured at 7 days after a first injection, an increased CD8 response is better seen with Siinfekl LT adjuvanted with AS than is seen with non-vectorised Siinfekl adjuvant or Siinfekl LT-X alone (FIG. 1A). This improvement is seen at doses as low as 0.1 μg of LT-X. The same is seen with Siinfekl LFn (FIG. 2A), and with Siinfekl-ExoA (FIG. 2A). However, this improvement is not seen when measured 15 days post 1^{st} dose (FIGS. 1B and 2B).

When looked at 7 days following a second injection, an improvement is again seen with Siinfekl LT (FIG. 1C), Siinfekl LFn and Siinfekl-ExoA (FIG. 2C) when combined with adjuvant system A, compared to either non-vectorized Siinfekl adjuvant or Siinfekl LFn or LT or ExoA without adjuvant.

FIG. 3 shows that when using full length ovalbumin protein as antigen, a better result is seen when using ovalbumin conjugated to either the LTB or the LTBcys vector in combination with an adjuvant than is seen with either conjugated ovalbumin alone or non-vectorised ovalbumin administered with Adjuvant system A. This improvement is seen at both 7 and 14 days post infection, and 7 days following second injection.

FIGS. 4A and 4B look at detection of CD8 cytokine-producing T cells. At least 2 cytokine are produced at both time point shown: 14 days after 1^{st} injection (FIG. 4A) and 6 days after 2^{nd} injection (FIG. 4B). Again it can be seen that the combination of adjuvant and vectorization shows a synergistic effect in comparison to vectorized antigen alone or to non-vectorized antigen plus adjuvant. This is again true with both different conjugation chemistries—direct conjugation of the antigen to the vector, or conjugation via a cysteine residue.

FIG. 4C looks at detection of CD4 cytokine-producing T cells (at least 2 cytokine are produced). Although in lesser extend, it is again shown that the combination of adjuvant and vectorization has a synergistic effect in comparison to vectorized antigen alone or to non-vectorized antigen plus adjuvant. This is again true with both different conjugation chemistries.

### Evaluation of the Immune Response Induced by a Composition Comprising Ova Conjugated to LT, Hepatitis B Surface Antigen (HBs) as Free Antigen, and Adjuvant System A

FIGS. 5-12 evaluate the immune response to two antigens—ova conjugated to LT, and yeast-produced and purified recombinant Hepatitis B surface protein included as free antigen in the same composition. The composition was adjuvanted with adjuvant system A. The whole adaptive immune response was examined, antibodies were measured against both antigens (FIG. 12) and tetramer read outs were taken (FIG. 5). In addition, CD4 and CD8 responses were measured at 7 and 14 days post 1^{St} injection and 7 days post second injection (FIGS. 6-11). Responses are shown as total cytokine (IFNg/TNFa/IL2) producing T cells.

The tetramer read outs show that siinfekl specific responses can be seen when HBs is present as free antigen, therefore confirming that the presence of free antigen does not interfere with the immune response to the conjugated antigen.

Cytokine responses were seen at all time points to both antigens, although the primary response was very low. As anticipated, Ova specific CD4 response was lower than the CD8 response. HBs and Ova specific T cell responses were both detectable in the secondary response measured at 7 days post 2^{nd} injection. A positive impact of the HBs antigen can be seen on the ova-specific T-Cell response induced by the adjuvanted vector.

Both antigens generate humoral responses measured 15 days post 2^{nd} injection. This shows that the presence of free or conjugated antigen does not impede with the immune response seen to the other antigen.

What is claimed is:

1. A vaccine composition comprising a non-live vector which targets the MHC class I pathway derived from a bacterial toxin or an immunologically functional derivative thereof but excluding those which bind the Gb3 receptor complexed with at least one first antigen and further comprising at least one second antigen and an adjuvant.

2. A vaccine composition according to claim 1 wherein the non-live vector is selected from the group consisting of: anthrax lethal factor (LF), P. aeruginosa exotoxin A, the B subunit from E. coli labile toxin (LT1), LT2, the cholera toxin (CT), the Bordatella Pertussis toxin (PT) a subtilase cytotoxin and the adenylate cyclase A from B. pertussis.

3. A vaccine composition according to claim 2 wherein the non-live vector is the B subunit from E. coli labile toxin (LT) or an immunologically functional equivalent thereof.

4. A vaccine composition as claimed in claim 1 wherein the adjuvant is selected from the group of metal salts, oil in water emulsions, Toll like receptor ligands, saponins or combinations thereof.

5. A vaccine composition as claimed in claim 4 wherein the adjuvant is a Toll like receptor ligand.

6. A vaccine composition as claimed in claim 5 wherein the toll like receptor ligand is an agonist.

7. A vaccine composition as claimed in claim 1, wherein the antigen and non-live vector which derived from a bacterial toxin or an immunologically functional derivative thereof are complexed together.

8. A vaccine composition as claimed in claim 1, wherein the antigen and non-live derived from a bacterial toxin or an immunologically functional derivative thereof are covalently attached.

9. A vaccine composition as claimed in claim 8 wherein the antigen and non-live vector derived from a bacterial toxin or an immunologically functional derivative thereof are joined as a fusion protein.

10. A vaccine composition as claimed in claim 1 wherein the adjuvant is selected from the group: metallic salts, a saponin, lipid A or derivative thereof, an alkyl glucosaminide phosphate, an immunostimulatory oligonucleotide or combinations thereof.

11. A vaccine composition as claimed in claim 10 wherein the saponin is presented in the form of a liposome, Iscom, or an oil in water emulsion.

12. A vaccine composition as claimed in claim 10 wherein the saponin is QS21.

13. A vaccine composition as claimed in claim 10, wherein the Lipid A derivative is selected from Monophosphoryl lipid A, 3 deacylated Monophosphoryl lipid A, OM 174, OM 197, OM 294.

14. A vaccine composition as claimed in claim 1 wherein the adjuvant is a combination of at least one representative from two of the following groups,
i) a saponin,
ii) a Toll-like receptor 4 ligand, and
iii) a Toll-Like receptor 9 ligand.
iv)

15. A vaccine composition as claimed in claim 14 wherein the saponin is QS21 and the toll like receptor 4 ligand is 3 deacylated monophosphoryl lipid A and the toll like receptor 9 is a CpG containing immunostimulatory oligonucleotide.

16. A vaccine composition as claimed in claim 1 wherein the first and second antigen are the same.

17. A vaccine composition as claimed in claim 16 wherein the antigen is selected from the group of antigens that provide immunity against the group of diseases selected from, intracellular pathogens or proliferative diseases.

18. A vaccine composition as claimed in claim 1 wherein the first antigen and the second antigen are different.

19. A vaccine composition as claimed in claim 18 wherein the first antigen is NS3 from HCV.

20. A vaccine composition as claimed in claim 19 wherein the second antigen is E1 from HCV.

21. A vaccine composition comprising a non-live vector derived from a bacterial toxin or an immunologically functional derivative thereof complexed with a first antigen and further comprising a second antigen and an adjuvant for use in medicine.

22. (canceled)

23. (canceled)

24. A method of treating or preventing disease comprising administering to a patient suffering from or susceptible to disease a vaccine composition according to claim 1.

25. A method for raising an antigen specific CD 8 immune response comprising the administration to a patient of a vaccine according to claim 1.

26. A process for the production of a vaccine according to claim 1 wherein a first antigen in combination with a non-live vector which targets the MHC class I pathway or an immunologically functional derivative thereof is admixed with an adjuvant and a second antigen.


SEQUENCE LISTING (6 sequences)

SEQ ID NO: 1 - DNA, 20 bp, Artificial Sequence
  Immunostimulatory oligonucleotide CpG 1826
tccatgacgttcctgacgtt

SEQ ID NO: 2 - DNA, 18 bp, Artificial Sequence
  Immunostimulatory oligonucleotide CpG 1758
tctcccagcgtgcgccat

SEQ ID NO: 3 - DNA, 30 bp, Artificial Sequence
  Immunostimulatory oligonucleotide
accgatgacgtcgccggtgacggcaccacg

SEQ ID NO: 4 - DNA, 24 bp, Artificial Sequence
  Immunostimulatory oligonucleotide CpG 2006
tcgtcgttttgtcgttttgtcgtt

SEQ ID NO: 5 - DNA, 20 bp, Artificial Sequence
  Immunostimulatory oligonucleotide CpG 1668
tccatgacgttcctgatgct

SEQ ID NO: 6 - DNA, 22 bp, Artificial Sequence
  Immunostimulatory oligonucleotide CpG 5456
tcgacgttttcggcgcgcgccg